(19) United States
(12) Patent Application Publication
(10) Pub. No.: US 2026/0283910 A1
(43) Pub. Date: Sep. 24, 2026

(54) Process for Producing a Zirconia Dental Article

(71) Applicant: Solventum Intellectual Properties Company, Maplewood, MN (US)

(72) Inventors: Gareth A. Hughes, St. Paul, MN (US); Yuxin Tong, Lake Elmo, MN (US); James D. Hansen, White Bear Lake, MN (US); Melissa A. Lackey, Woodbury, MN (US); Martin Goetzinger, Vilgertshofen (DE)

(21) Appl. No.: 19/474,333

(22) PCT Filed: Apr. 11, 2024
(86) PCT No.: PCT/IB2024/053571

§ 371 Date: Oct. 10, 2025

(30) Foreign Application Priority Data
May 10, 2023 (EP) 23172501.1

(51) Int. Cl. A61K 6/818; A61K 6/16; A61K 6/17; A61K 6/62; A61K 6/78; B33Y 10/00; B33Y 70/10; B33Y 80/00; C04B 35/622; C04B 35/624; C04B 35/626; C04B 35/64

(52) U.S. Cl. CPC A61K 6/818; A61K 6/16; A61K 6/17; A61K 6/62; A61K 6/78; B33Y 10/00; B33Y 70/10; B33Y 80/00; C04B 35/62222; C04B 35/624; C04B 35/62645; C04B 35/6269; C04B 35/64; C04B 2235/3244

(57) ABSTRACT

The invention relates to a process of producing a dental zirconia article, the process comprising the steps of processing a radiation-curable zirconia sol A as construction material in an additive-manufacturing process comprising a radiation-curing step to obtain a zirconia gel article, the zirconia gel article having the shape of a dental article with an outer and an inner surface, applying a radiation-curable zirconia sol Bₓ on at least a portion of the outer surface of the zirconia gel article, radiation-curing the radiation-curable zirconia sol Bₓ of step (b), transforming the zirconia gel article into a zirconia aerogel article, the radiation-curable zirconia sol A being same or different from the radiation-curable zirconia sol Bₓ with respect to chemical composition and/or physical properties. The invention also relates to a dental ceramic zirconia article obtainable by the process described in any of the proceeding claims.

### FIELD OF THE INVENTION

The invention relates to a process for producing a dental zirconia article, a dental zirconia article obtainable by such a process and a kit of parts comprising two different zirconia sols to be processed in such a process.

### BACKGROUND

The processing of ceramic sols by additive-manufacturing techniques is generally known.
US 2015/0223917 A1 (Herrmann et al.) relates to a kit, comprising: a coloring solution; a porous zirconia article; optionally application equipment; wherein the coloring solution comprises: cation(s) of coloring agent(s) in an amount above about 0.05 mol per 1 coloring solution, solvent(s) for the ion(s); optionally complexing agent(s); optionally thickening agent(s); optionally organic marker substance(s); and optionally additive(s); wherein the porous zirconia article showing a N₂ adsorption and desorption of isotherm type IV according to IUPAC classification and a BET surface from about 10 to about 200 m²/g.
WO 01/13815 A1 (Feenstra) describes a method for making a dental element by a 3-dim printing technique. As curable material preferably a nanomeric material consisting of nanomeric inorganic solid particles having polymerizable organic groups at their surface is used. After the printing process, the dental element is typically subject to a thermal post-treatment between 6° and 150° C. to complete curing. Instead thereof, or supplemental thereof, a thermal densification is accomplished wherein the dental element is heated to a temperature of at least 250° C.
U.S. Pat. No. 10,759,707 (Mayr et al.) relates to a process for producing a ceramic article, the process comprising the steps of providing a printing sol, the printing sol comprising solvent, nano-sized particles, radiation curable monomer(s) and photo-initiator, the printing sol having a viscosity of less than 500 mPa*s at 23° C., processing the printing sol as construction material in an additive manufacturing process to obtain a 3-dim article being in a gel state, the 3-dim article having a Volume A, transferring the 3-dim article being in a gel state to a 3-dim article being in an aerogel state, heat treating the 3-dim article to obtain a sintered 3-dim ceramic article, the ceramic article having a Volume F, Volume A of the 3-dim article in a gel state being more than 500% of Volume F of the ceramic article in its sintered state. Described is also a ceramic article obtainable according to such a process, which may have the shape of a dental or orthodontic article.
US 2021/32283 A1 (Jans et al.) describes a glazing composition suitable for glazing the surface of a dental zirconia article. The glazing composition comprises a liquid, glass particles and hydrophilic silica nano-particles.

### SUMMARY OF INVENTION

However, there is still a need for a process for producing a highly aesthetic dental zirconia article.

The process should allow the production of customized dental articles which can be used by the practitioner without the need for further modification steps like milling.

Ideally, the customized dental article should have a shiny surface.

In one aspect the present invention features a process of producing a dental zirconia article, the process comprising the steps of
(a) processing a radiation-curable zirconia sol A as construction material in an additive-manufacturing process comprising a radiation-curing step to obtain a zirconia gel article, the zirconia gel article having the shape of a dental article with an outer and an inner surface,
(b) applying a radiation-curable zirconia sol Bₓ on at least a portion of the outer surface of the zirconia gel article,
(c) radiation-curing the radiation-curable zirconia sol Bₓ of step (b),
(d) transforming the zirconia gel article into a zirconia aerogel article,
the radiation-curable zirconia sol A being same or different from the radiation-curable zirconia sol Bₓ with respect to chemical composition and/or physical properties as described in the claims and the present text.

In another aspect, the invention relates to the dental zirconia article obtainable or obtained by the process described in the claims and the present text.

Unless defined differently, for this description the following terms shall have the given meaning:

A “hardenable or curable or polymerizable component” is any component which can be cured or solidified in the presence of a photo-initiator by radiation-induced polymerization. A hardenable component may contain only one, two, three or more polymerizable groups. Typical examples of polymerizable groups include unsaturated carbon groups, such as a vinyl group being present i.a. in a (methyl)acrylate group.

As used herein, “(meth)acryl” is a shorthand term referring to “acryl” and/or “methacryl”. For example, a “(meth)acryloxy” group is a shorthand term referring to either an acryloxy group (i.e., CH₂═CH—C(O)—O—) and/or a methacryloxy group (i.e., CH₂═C(CH₃)—C(O)—O—).

As used herein, “hardening” or “curing” a composition are used interchangeably and refer to polymerization and/or crosslinking reactions including, for example, photo-polymerization reactions and chemical-polymerization techniques (e. g., ionic reactions or chemical reactions forming radicals effective to polymerize ethylenically unsaturated compounds) involving one or more materials included in the composition.

“Dental article” means an article which is to be used in the dental or orthodontic field, especially as or for producing a dental restoration. A dental article has typically two different surface portions, an outer surface and an inner surface. The outer surface is the surface which is typically not in permanent contact with the surface of a tooth. In contrast thereto, the inner surface is the surface which is used for attaching or fixing the dental article to a tooth. If the dental article has the shape of a dental crown or dental veneer, the inner surface has typically a concave shape, whereas the outer surface has typically a convex shape. A dental article should not contain components which are detrimental to the patient's health and thus free of hazardous and toxic components being able to migrate out of the dental or orthodontic article.

“Dental restoration” means dental articles which are used for restoring a tooth to be treated. Examples of dental restorations include crowns, bridges, inlays, onlays, veneers, and parts thereof.

Examples of orthodontic articles include brackets, buccal tubes, cleats and buttons and parts thereof.

“Sol” refers to a continuous liquid phase containing discrete particles having sizes in a range of 1 nm to 100 nm or 1 nm to 50 nm, a so called “colloidal solution”. The sols described in the present text are translucent and do show a so-called “Tyndall effect” or “Tyndall scattering”. The size of the particles is below the wavelength of the visible light (400 to 700 nm).

“Coloring ions” shall mean ions which have an absorption in the spectrum visible to the human eye (e.g., from about 380 to about 780 nm), which results in a colored solution (visible to the human eye), if the coloring ions are dissolved in water (e.g., about 0.6 mol/I) and/or cause a coloring effect in the zirconia article containing coloring ions.

A composition can be characterized as “transparent” if a beam of visible light (380 to 780 nm) is not scattered by the composition and cannot be observed by side view (i.e., no Tyndall effect). However, the intensity of the penetrating beam of visible light in direction of the beam may be weakened due to absorption of the light by the coloring ions. A transparent composition lets light pass through according to Snell's law (classical law of refraction). So, a picture can be seen in its details through a platelet of a transparent material.

A translucent composition lets light partially permeate through although it is not fully transparent, i.e. showing a significant volume scattering of the transmitted light. The reciprocal property of translucency is opacity (O). O=1/T=I/I0 (T=Transmission, I=Intensity of permeated light, I=Intensity of light before permeation). So, opacity values less than about 0.9 for a 1 mm thick platelet with a diameter of 15 mm are regarded as translucent (e.g., for a measurement with a Color i7 device, X-Rite corporation USA, measurement mode: remission contrast ratio). Opacity can be measured by various means: in transmission, in remission, and in remission using the contrast ratio method.

A “particle” means a substance being a solid having a shape which can be geometrically determined. The shape can be regular or irregular. Particles can typically be analysed with respect to e.g. particle size and particle size distribution.

The term “primary particle size” refers to the size of a non-associated single particle, which is considered to be a primary particle. X-ray diffraction (XRD) is typically used to measure the primary particle size.

A dental ceramic article is classified as “pre-sintered” if the dental ceramic article has been treated with heat (temperature range from 900 to 1,100° C.) for 1 to 3 h to such an extent that the raw breaking resistance of the dental ceramic measured according to the “punch on three ball test” ISO 6872 is within a range of 15 to 55 MPa or 30 to 50 MPa. A pre-sintered dental ceramic article usually has a porous structure and its density (usually 3.0 g/cm³ for a 3 mol % yttrium stabilized zirconia ceramic) is less compared to a completely sintered dental ceramic framework (usually 6.1 g/cm³ for a 3 mol % yttrium stabilized zirconia ceramic).

“Ceramic zirconia article” shall mean a 3-dimensional article wherein at least one the x, y, z dimension is at least about 5 mm, the article being comprised of at least 80 wt. % or at least 90 wt. % zirconia.

A “monolithic ceramic zirconia article” shall mean an article the surface of which is not coated with a material or composition other than a zirconia material.

“Ceramic” means an inorganic non-metallic material that is produced by application of heat. Ceramics are usually hard, porous and brittle and, in contrast to glasses or glass ceramics, display an essentially purely crystalline structure.

“Crystalline” means a solid composed of atoms arranged in a pattern periodic in three dimensions (i.e., has long range crystal structure as determined by X-ray diffraction). Crystal structures include tetragonal, monocline, cubic zirconia and mixtures thereof.

“Diafiltration” is a technique that uses ultrafiltration membranes to completely remove, replace, or lower the concentration of salts or solvents from solutions containing organic molecules. The process selectively utilizes permeable (porous) membrane filters to separate the components of solutions and suspensions based on their molecular size.

The term “aerogel” shall mean a three-dimensional low density solid (i.e., less than 20% of the density of an article having a porosity of less than 0.1 vol. %). An aerogel is a porous material derived from a gel, in which the liquid component of the gel has been replaced with a gas. The solvent removal is often done under supercritical conditions. During this process the network does not substantially shrink and a highly porous, low-density material can be obtained.

“Density” means the ratio of mass to volume of an object. The unit of density is typically g/cm³. The density of an object can be calculated e.g., by determining its volume (e.g., by calculation or applying the Archimedes principle or method) and measuring its mass.

The term “tubular reactor” refers to the portion of a continuous hydrothermal reactor system that is heated (i.e., the heated zone). The tubular reactor can be in any suitable shape. The shape of the tubular reactor is often selected based on the desired length of the tubular reactor and the method used to heat the tubular reactor. For example, the tubular reactor can be straight, U-shaped, or coiled. The interior potion of the tubular reactor can be empty or can contain baffles, balls, or other known mixing techniques.

The term “calcining” refers to a process of heating a solid material to drive off at least 90 percent by weight of volatile chemically bond components (e.g., organic components) (vs., for example, drying, in which physically bonded water is driven off by heating). Calcining is done at a temperature below a temperature needed to conduct a pre-sintering step.

The terms “sintering” or “firing” are used interchangeably. A pre-sintered ceramic article shrinks during a sintering step, that is, if an adequate temperature is applied. The sintering temperature to be applied depends on the ceramic material chosen. For zirconia based ceramics a typical sintering temperature range is 1,100° C. to 1,400° C. Sintering typically includes the densification of a porous material to a less porous material (or a material having less cells) having a higher density, in some cases sintering may also include changes of the material phase composition (for example, a partial conversion of an amorphous phase toward a crystalline phase).

“Additive manufacturing” means processes used to make 3-dimensional articles. An example of an additive manufacturing technique is stereolithography (SLA) in which successive layers of material are laid down under computer control. The articles can be of almost any shape or geometry and are produced from a 3-dimensional model or other electronic data source.

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g., the term “additive(s)” means one additive and more additives (e.g., 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not willfully added to the composition either as such or in combination with other components or ingredient of other components. A composition being essentially free of a certain component usually does not contain that component at all. However, sometimes the presence of a small amount of the said component is not avoidable e.g., due to impurities contained in the raw materials used.

### BRIEF DESCRIPTION OF FIGURES

FIG. 1 demonstrates the volume shrinkage occurring if a printed gel article is transformed to an aerogel article, which is then pre-sintered and fully sintered (from left to right).

FIG. 2 shows dental zirconia veneers prepared according to the invention (the two samples on the right) and dental zirconia veneers prepared not according to the invention (the two samples on the left).

### DETAILED DESCRIPTION

It has been found that the invention described in the text has a couple of advantageous properties.

The invention facilitates an additive, mould-free approach to final part fabrication as compared to existing moulding and/or subtractive processes, e.g. machining a ceramic article out of a mill blank.

Currently, the individualization of a dental zirconia article is typically done on the sintered dental zirconia article using a glazing and/or coloring composition which chemically differs from the subjacent zirconia material. Usually, the glazing materials are also less durable than the zirconia material.

This may result in an undesirable aesthetic change of appearance of the dental article over time. Further, such a structure may also cause increased wear on the opposing tooth.

Further, the individualization of the surface of the sintered dental zirconia article is labour-intensive and due to its small size requires specific skills and equipment.

According to the present invention the glazing and coloring, if desired, is done with essentially the same zirconia material used for producing the dental article.

As the glazing material undergoes the same process steps as the subjacent zirconia material, the resulting dental article is more durable.

A dental article obtained by such a process and inserted in the mouth of a patient does also not show excess wear on the opposing tooth.

The process described in the present text allows the production of the final desired shape already during the additive-manufacturing step.

The coloring and glazing can be performed on the as-printed zirconia gel using the same or similar materials as the zirconia gel itself.

This results in a high quality, glazed-like surface that is also highly durable, and can impart different colors depending on the dopant composition of the coating liquid.

Unexpectedly, the application of a radiation-curable sol already in an early state resulted in a smooth and glossy surface of the dental zirconia article after sintering while a dental article not having been treated in this way has a matte surface, even when cleaned.

Additionally, poor cleaning often results in a hazy, white surface, but the excess sol coating in this case does not turn white.

Further, as the coloring and/or glazing is done on the surface of the dental article being in a gel state, the coloring and/or glazing can more easily infiltrate the gel material of the dental article.

The fact that the size of the dental zirconia article being in a gel state is much bigger compared to the size of the dental zirconia article after sintering, facilitates the individualization of the surface of the dental zirconia article.

The process described in the present text allows the production of sol-gel bodies with a resolution of 25 μm in z-direction and 40 μm×40 μm in x-y direction (=pixel size) resulting in a ceramic body (after sintering) with a resolution of e.g. 12.5 μm in z-direction and 20 μm×20 μm in x-y direction.

In a first step a radiation-curable zirconia sol A is processed as a construction material in an additive-manufacturing process comprising a radiation-curing step to obtain a zirconia gel article. The radiation-curable zirconia sol A is also referred to as printing sol.

The zirconia gel article has the shape of a dental article with an outer and an inner surface, that is, the zirconia gel article has a 3-dimensional shape.

The radiation-curable zirconia sol A is a dispersion of crystalline zirconia particles in a liquid.

Sols which were found to be generally suitable are described e.g. in WO 2013/055432 A1 (3M) relating to aerogels, calcined articles and crystalline articles comprising zirconia and methods of making the same. U.S. Pat. No. 7,429,422 (Davidson et al.) also describes methods of making zirconia-based sols, which can be used. Further sols which can be used are described in WO 2016/140840 A1 (3M). The above references are herewith incorporated by reference.

The radiation-curable zirconia sol A comprises
crystalline zirconia particles, preferably in an amount of 25 to 65 wt. %,
a crystal phase stabilizer component, preferably in an amount of 2 to 8 mol % calculated as oxide with respect to the total crystalline oxide particles,
a radiation-curable component, preferably in an amount of 2 to 30 wt. %,
a photo-initiator, preferably in an amount of 0.01 to 3 wt. %,
a liquid, preferably in an amount of 20 to 70 wt. %,
optionally coloring ions, preferably in an amount of 0 to 2 wt. %,
optionally inhibitor components, preferably in an amount of 0 to 0.5 wt. %, wt. % with respect to the weight of the radiation-curable zirconia sol A.

The crystal phase stabilizer component is typically contained in the crystalline zirconia particles and the optional coloring ions can be contained in the crystalline zirconia particles and/or be present as a separate component.

The crystalline zirconia particles in the zirconia sol A typically have a primary particle size in a range of 2 nm to 50 nm (in some embodiments, 5 nm to 50 nm, 2 nm to 25 nm, 5 nm to 25 nm, 2 nm to 15 nm, or even 5 nm to 15 nm).

The crystalline zirconia particles are typically present in the following amounts: at least 25 or at least 30 or at least 35 wt. %; at most 65 or at most 60 or at most 55 wt. %; 25 to 65, or 30 to 60, or 35 to 55 wt. %; wt. % with respect to the weight of the printing sol.

Crystal phase stabilizing components which can be used include oxides of Ce, Mg, Ca, Y, La, rare earth elements and combinations thereof, wherein the use of Y and La is sometimes preferred.

The crystal phase stabilizer component calculated as oxide is typically present in the following amounts: at least 2 or at least 3 or at least 4 mol %; at most 8 or at most 7 or at most 6 mol %; 2 to 8, or 3 to 7, or 4 to 6 mol %; mol % with respect to the total crystalline oxide particles.

The radiation-curable component being present in the printing sol can be described as first, second, third, etc. monomer.

The zirconia particles which have been surface-modified with a polymerizable agent can be polymerized, if desired, to provide a composition comprising crosslinked zirconia particles.

A first monomer can function as a polymerizable surface modification agent. Multiple first monomers can be used.

Surface modification agents may be represented by the formula A-B, where the A group is capable of attaching to the surface of a zirconia-based particle and the B group is radiation curable.

Group A can be attached to the surface of the zirconia-based particle by adsorption, formation of an ionic bond, formation of a covalent bond, or a combination thereof.

Examples for Group A include acidic moieties (like carboxylic acid groups, phosphoric acid groups, sulfonic acid groups and anions thereof) and silanes.

Group B comprises a radiation curable moiety.

Examples for Group B include vinyl, in particular acryl or methacryl moieties.

Suitable surface modifying agents comprise polymerizable carboxylic acids and/or anions thereof, polymerizable sulfonic acids and/or anions thereof, polymerizable phosphoric acids and/or anions thereof, and polymerizable silanes. Suitable surface modification agents are further described, for example, in WO 2009/085926 A1 (Kolb et al.).

An example of a radically polymerizable surface modifier is a polymerizable surface modification agent comprising an acidic moiety or anion thereof, e.g. a carboxylic acid group.

Exemplary acidic radically polymerizable surface modifiers include acrylic acid, methacrylic acid, beta-carboxyethyl acrylate, and mono-2-(methacryloxyethyl)succinate.

Exemplary radically polymerizable surface modifiers can be reaction products of hydroxyl-containing polymerizable monomers with cyclic anhydrides such as succinic anhydride, maleic anhydride and phthalic anhydride. Exemplary polymerization hydroxyl-containing monomers include hydroxyethyl acrylate, hydroxyethyl methacrylate, hydroxypropyl acrylate, hydroxypropyl methacrylate, hydroxybutyl acrylate, and hydroxybutyl methacrylate. Acryloxy and methacryloxy functional polyethylene oxide and polypropylene oxide may also be used as the polymerizable hydroxyl-containing monomers.

An exemplary radically polymerizable surface modifier for imparting both polar character and reactivity to the zirconia-containing nanoparticles is mono(methacryloxypolyethyleneglycol) succinate.

Another example of a radically polymerizable surface modifier is a polymerizable silane.

Exemplary polymerizable silanes include methacryloxyalkyltrialkoxysilanes or acryloxyalky-Itrialkoxysilanes (e.g., 3-methacryloxypropyltrimethoxysilane, 3-acryloxypropyltrimethoxy-silane, and 3-(methacryloxy)propyltriethoxysilane); methacryloxyalkylalkyldialkoxysilanes or acryloxyalkylalkyldialkoxysilanes (e.g., 3-(methacryloxy)propylmethyldimethoxysilane and 3-(acryloxypropyl)methyldimethoxysilane); methacryloxyalkyldialkylalkoxysilanes or acyrloxyalkyldialkylalkoxysilanes (e.g., 3-(methacryloxy)propyldimethylethoxysilane); mercaptoalkyltrialkoxylsilanes (e.g., 3-mercaptopropyltrimethoxysilane); aryltrialkoxy-silanes (e.g., styrylethyltrimethoxysilane); vinylsilanes (e.g., vinylmethyldiacetoxysilane, vinyldimethylethoxysilane, vinylmethyldiethoxysilane, vinyltrimethoxysilane, vinyltriethoxysilane, vinyltriacetoxysilane, vinyltriisopropoxysilane, and vinyltris(2-methoxy-ethoxy)silane).

According to one embodiment, the printing sol described in the present text comprises one or more second monomers comprising at least two radiation curable moieties. Those second monomer(s) may act as crosslinker(s) during the gel-forming step.

Any suitable second monomerthat does not have a surface modification group can be used. That is, the optional second monomer does not have a carboxylic acid group or a silyl group. The second monomers are often polar monomers (e.g., non-acidic polar monomers), monomers having a plurality of polymerizable groups, alkyl (meth)acrylates and mixtures thereof.

The presence of the monomer having a plurality of polymerizable groups tends to enhance the strength of the gel composition formed when the printing sol is polymerized.

The number of polymerizable groups can be in a range of 2 to 6 or even higher. In many embodiments, the number of polymerizable groups is in a range of 2 to 5 or 2 to 4. The polymerizable groups are typically (meth)acryloyl groups.

Exemplary monomers with two (meth)acryloyl groups include 1,2-ethanediol diacrylate, 1,3-propanediol diacrylate, 1,9-nonanediol diacrylate, 1,12-dodecanediol diacrylate, 1,4-butanediol diacrylate, 1,6-hexanediol diacrylate, butylene glycol diacrylate, bisphenol A diacrylate, diethylene glycol diacrylate, triethylene glycol diacrylate, tetraethylene glycol diacrylate, tripropylene glycol diacrylate, polyethylene glycol diacrylate, polypropylene glycol diacrylate, polyethylene/polypropylene copolymer diacrylate, polybutadiene di(meth)acrylate, propoxylated glycerin tri(meth)acrylate, and neopentylglycol hydroxypivalate diacrylate modified caprolactone.

Exemplary monomers with three or four (meth)acryloyl groups include trimethylolpropane triacrylate (e.g., commercially available under the trade designation TMPTA-N from Cytec Industries, Inc. (Smyrna, GA, USA) and under the trade designation SR-351 from Sartomer (Exton, PA, USA)), pentaerythritol triacrylate (e.g., commercially available under the trade designation SR-444 from Sartomer), ethoxylated (3) trimethylolpropane triacrylate (e.g., commercially available under the trade designation SR-454 from Sartomer), ethoxylated (4) pentaerythritol tetraacrylate (e.g., commercially available under the trade designation SR-494 from Sartomer), tris(2-hydroxyethylisocyanurate)triacrylate (e.g., commercially available under the trade designation SR-368 from Sartomer), a mixture of pentaerythritol triacrylate and pentaerythritol tetraacrylate (e.g., commercially available from Cytec Industries, Inc., under the trade designation PETIA with an approximately 1:1 ratio of tetraacrylate to triacrylate and under the trade designation PETA-K with an approximately 3:1 ratio of tetraacrylate to triacrylate), pentaerythritol tetraacrylate (e.g., commercially available under the trade designation SR-295 from Sartomer), and di-trimethylolpropane tetraacrylate (e.g., commercially available under the trade designation SR-355 from Sartomer).

Exemplary monomers with five or six (meth)acryloyl groups include dipentaerythritol pentaacrylate (e.g., commercially available under the trade designation SR-399 from Sartomer) and a hexa-functional urethane acrylate (e.g., commercially available under the trade designation CN975 from Sartomer).

In some embodiments, the optional second monomer is a polar monomer. As used herein, the term “polar monomer” refers to a monomer having a free radical polymerizable group and a polar group. The polar group is typically non-acidic and often contains a hydroxyl group, a primary amido group, a secondary amido group, a tertiary amido group, an amino group, or an ether group (i.e., a group containing at least one alkylene-oxy-alkylene group of formula —R—O—R— where each R is an alkylene having 1 to 4 carbon atoms).

Suitable optional polar monomers having a hydroxyl group include, but are not limited to, hydroxyalkyl (meth)acrylates (e.g., 2-hydroxyethyl (meth)acrylate, 2-hydroxypropyl(meth)-acrylate, 3-hydroxypropyl (meth)acrylate, and 4-hydroxybutyl (meth)acrylate), and hydroxyalkyl (meth)acrylamides (e.g., 2-hydroxyethyl (meth)acrylamide or 3-hydroxypropyl (meth)acrylamide), ethoxylated hydroxyethyl (meth)acrylate (e.g., monomers commercially available from Sartomer (Exton, PA, USA) underthe trade designation CD570, CD571, and CD572), and aryloxy substituted hydroxyalkyl (meth)acrylates (e.g., 2-hydroxy-2-phenoxy-propyl (meth)acrylate).

Exemplary polar monomers with a primary amido group include (meth)acrylamide. Exemplary polar monomers with secondary amido groups include, but are not limited to, N-alkyl (meth)acrylamides such as N-methyl (meth)acrylamide, N-ethyl (meth)acrylamide, N-isopropyl (meth)acrylamide, N-tert-octyl(meth)acrylamide, and N-octyl(meth)acrylamide. Exemplary polar monomers with a tertiary amido group include, but are not limited to, N-vinyl caprolactam, N-vinyl-2-pyrrolidone, (meth)acryloyl morpholine, and N,N-dialkyl (meth)acrylamides such as N,N-dimethyl (meth)acrylamide, N,N-diethyl(meth)acrylamide, N,N-dipropyl (meth)acrylamide, and N,N-dibutyl(meth)acrylamide.

Polar monomers with an amino group include various N,N-dialkylaminoalkyl (meth)acrylates and N,N-dialkylaminoalkyl (meth)acrylamides. Examples include, but are not limited to, N,N-dimethyl aminoethyl (meth)acrylate, N,N-dimethylaminoethyl (meth)acrylamide, N,N-dimethylaminopropyl (meth)acrylate, N,N-dimethylaminopropyl (meth)acrylamide, N,N-diethylaminoethyl (meth)acrylate, N,N-diethylaminoethyl(meth)acrylamide, N,N-diethylaminopropyl (meth)acrylate, and N,N-diethylaminopropyl (meth)acrylamide.

Exemplary polar monomers with an ether group include, but are not limited to, alkoxylated alkyl (meth)acrylates such as ethoxyethoxyethyl (meth)acrylate, 2-methoxyethyl (meth)acrylate, and 2-ethoxyethyl (meth)acrylate; and poly(alkylene oxide)(meth)acrylates such as poly(ethylene oxide)(meth)acrylates, and poly(propylene oxide)(meth)acrylates. The poly(alkylene oxide) acrylates are often referred to as poly(alkylene glycol)(meth)acrylates. These monomers can have any suitable end group such as a hydroxyl group or an alkoxy group. For example, when the end group is a methoxy group, the monomer can be referred to as methoxy poly(ethylene glycol)(meth)acrylate.

Suitable alkyl (meth)acrylates that can be used as a second monomer can have an alkyl group with a linear, branched, or cyclic structure. Examples of suitable alkyl (meth)acrylates include, but are not limited to, methyl (meth)acrylate, ethyl (meth)acrylate, n-propyl (meth)acrylate, isopropyl (meth)acrylate, n-butyl (meth)acrylate, isobutyl (meth)acrylate, n-pentyl(meth)acrylate, 2-methylbutyl(meth)acrylate, n-hexyl(meth)acrylate, cyclohexyl (meth)acrylate, 4-methyl-2-pentyl(meth)acrylate, 2-ethylhexyl (meth)acrylate, 2-methylhexyl (meth)acrylate, n-octyl (meth)acrylate, isooctyl (meth)acrylate, 2-octyl(meth)acrylate, isononyl (meth)acrylate, isoamyl(meth)acrylate, 3,3,5-trimethylcyclohexyl (meth)acrylate, n-decyl(meth)acrylate, isodecyl(meth)acrylate, isobornyl (meth)acrylate, 2-propylheptyl(meth)acrylate, isotridecyl(meth)acrylate, isostearyl (meth)acrylate, octadecyl(meth)acrylate, 2-octyldecyl(meth)acrylate, dodecyl (meth)acrylate, lauryl (meth)acrylate, and heptadecanyl(meth)acrylate.

In certain embodiments the second monomer(s) can be characterized by the following features alone or in combination:
soluble in the liquid contained in the sol;
bearing at least one or two or three radiation-curable moieties;
bearing radiation curable moieties selected from vinyl, acryl or methacryl moieties;
molecular weight: 70 to 5,000 g/mol; or 70 to 1,000 g/mol; or 100 to 500 g/mol.

Using radiation curable component(s) as described above having a molecular weight in the above range facilitates the provision of a sol having the desired viscosity. Lower molecular weight components are typically also better soluble than high molecular weight components.

The radiation-curable component is typically present in the following amounts: at least 2 or at least 5 or at least 10 wt. %; at most 30 or at most 25 or at most 20 wt. %; 2 to 30 or 5 to 25 or 10 to 20 wt. %; wt. % with respect to the weight of the printing sol.

The printing sol described in the present text comprises one or more photo-initiator(s).

The nature and structure of the photo-initiator is not particularly limited, either, unless the desired result cannot be achieved.

In certain embodiments the photo-initiator(s) can be characterized by the following features alone or in combination: soluble in the liquid contained in the sol; radiation absorption: within a range of 200 to 500 nm or 300 to 450 nm.

The photo-initiator should be able to start or initiate the curing or hardening reaction of the radiation curable component(s) being present in the printing sol.

In certain embodiments the following classes of photo-initiator(s) can be used: a) two-component system where a radical is generated through abstraction of a hydrogen atom from a donor compound; b) one component system where two radicals are generated by cleavage.

Examples of photo-initiators according to type (a) typically contain a moiety selected from benzophenone, xanthone or quinone in combination with an aliphatic amine.

Examples of photo-initiators according to type (b) typically contain a moiety selected from benzoin ether, acetophenone, benzoyl oxime or acyl phosphine.

Exemplary UV initiators include 1-hydroxycyclohexyl benzophenone (available under the trade designation “IRGACURE 184” (previously from Ciba Specialty Chemicals Corp., Tarrytown, NY), 4-(2-hydroxyethoxy)phenyl-(2-hydroxy-2-propyl) ketone (available under the trade designation “IRGACURE 2529” (previously from Ciba Specialty Chemicals Corp.), 2-hydroxy-2-methylpropiophenone (available under the trade designation “DAROCURE D111” previously from Ciba Specialty Chemicals Corp.) and bis(2,4,6-trimethylbenzoyl)-phenylphosphineoxide (available under the trade designation “IRGACURE 819” previously from Ciba Specialty Chemicals Corp.).

The photo-initiator is typically present in the following amounts: at least 0.01 or at least 0.05 or at least 0.08 wt. %; at most 3 or at most 2 or at most 1 wt. %; 0.01 to 3 or 0.05 to 2 or 0.08 to 1 wt. %; wt. % with respect to the weight of the printing sol.

The nature and structure of the liquid is not particularly limited unless the desired result cannot be achieved. In certain embodiments the solvent can be characterized by the following features alone or in combination:
a) boiling point: above 100 or above 120 or above 150° C.;
b) molecular weight: 25 to 300 g/mol;
c) viscosity: 0.2 to 10 mPa*s (23° C.);
d) miscible with water;
e) soluble in supercritical carbon dioxide or liquid carbon dioxide.

A combination of the following features is sometimes preferred: a) and b), or a), b) and c), or a), b), c) and d).

Using a liquid with a boiling point above 100° C. or 150° C. can be beneficial for reducing the evaporation of the liquid during the process.

Using a liquid with a molecular weight and/or viscosity in the above range can be beneficial as it helps in adjusting the viscosity of the printing sol. The molecular weight can also affect the diffusion constant and how easily the liquid can be removed.

Using a mixture of different liquids can be beneficial as it allows to adjust viscosity or post processing properties, e.g. removal of excess sol after printing.

The liquid should also be easily removable during the further processing steps needed for the realization of a ceramic article.

Further, the liquid should not interfere with or negatively influence the polymerization of the radiation-curable components being present in the sol.

In this respect, using liquid not bearing polymerizable moieties can be beneficial.

To enhance the dissolving capability or property of the liquid, the liquid typically bears one or more polar moieties, including ether, alcohol or carboxy moieties.

According to one embodiment, the liquid is often a glycol or polyglycol, mono-ether glycol or mono-ether polyglycol, di-ether glycol or di-ether polyglycol, ether ester glycol or ether ester polyglycol, carbonate, amide, or sulfoxide (e.g., dimethyl sulfoxide).

Suitable glycols or polyglycols, mono-ether glycols or mono-ether polyglycols, di-ether glycols or di-ether polyglycols, and ether ester glycols or ether ester polyglycols are often of the following Formula (I).

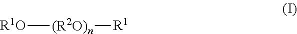

In Formula (I), each R¹ independently is hydrogen, alkyl, aryl, or acyl. Suitable alkyl groups often have 1 to 10 carbon atoms, 1 to 6 carbon atoms, or 1 to 4 carbon atoms. Suitable aryl groups often have 6 to 10 carbon atoms and are often phenyl or phenyl substituted with an alkyl group having 1 to 4 carbon atoms. Suitable acyl groups are often of formula —(CO)R^{a} where R^{a} is an alkyl having 1 to 10 carbon atoms, 1 to 6 carbon atoms, 1 to 4 carbon atoms, 2 carbon atoms, or 1 carbon atom. The acyl is often an acetyl group (—(CO)CH₃). In Formula (I), each R² is typically ethylene or propylene. The variable n is at least 1 and can be in a range of 1 to 10, 1 to 6, 1 to 4, or 1 to 3.

Glycols or polyglycols of Formula (I) have two R¹ groups equal to hydrogen. Examples of glycols include, but are not limited to, ethylene glycol, propylene glycol, diethylene glycol, dipropylene glycol, triethylene glycol, and tripropylene glycol.

Mono-ether glycols or mono-ether polyglycols of Formula (I) have a first R¹ group equal to hydrogen and a second R¹ group equal to alkyl or aryl. Examples of mono-ether glycols or mono-ether polyglycols include, but are not limited to, ethylene glycol monohexyl ether, ethylene glycol monophenyl ether, propylene glycol monobutyl ether, diethylene glycol monomethyl ether, diethylene glycol monoethyl ether, diethylene glycol monopropyl ether, diethylene glycol monobutyl ether, diethylene glycol monohexyl ether, dipropylene glycol monomethyl ether, dipropylene glycol monoethyl ether, dipropylene glycol monopropyl ether, triethylene glycol monomethyl ether, triethylene glycol monoethyl ether, triethylene glycol monobutyl ether, tripropylene glycol monomethyl ether, and tripropylene glycol monobutyl ether.

Di-ether glycols or di-ether polyglycols of Formula (I) have two R¹ group equal to alkyl or aryl. Examples of di-ether glycols or di-ether polyglycols include, but are not limited to, ethylene glycol dipropyl ether, ethylene glycol dibutyl ether, dipropylene glycol dibutyl ether, diethylene glycol dimethyl ether, diethylene glycol diethyl ether, triethylene glycol dimethyl ether, tetraethylene glycol dimethyl ether, and pentaethylene glycol dimethyl ether.

Ether ester glycols or ether ester polyglycols of Formula (I) have a first R¹ group equal to an alkyl or aryl and a second R¹ group equal to an acyl. Examples of ether ester glycols or ether ester polyglycols include, but are not limited to, ethylene glycol butyl ether acetate, diethylene glycol butyl ether acetate, and diethylene glycol ethyl ether acetate.

Other suitable organic solvents are carbonates of Formula (II).

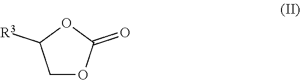

In Formula (II), R³ is hydrogen or an alkyl such as an alkyl having 1 to 4 carbon atoms, 1 to 3 carbon atoms, or 1 carbon atom. Examples include ethylene carbonate and propylene carbonate.

Yet other suitable organic solvents are amides of Formula (III).

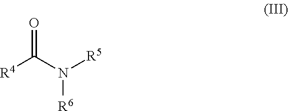

In Formula (III), group R⁴ is hydrogen, alkyl, or combines with R⁵ to form a five-membered ring including the carbonyl attached to R⁴ and the nitrogen atom attached to R⁵. Group R⁵ is hydrogen, alkyl, or combines with R⁴ to form a five-membered ring including the carbonyl attached to R⁴ and the nitrogen atom attached to R⁵. Group R⁶ is hydrogen or alkyl. Suitable alkyl groups for R⁴, R⁵, and R⁶ have 1 to 6 carbon atoms, 1 to 4 carbon atoms, 1 to 3 carbon atoms, or 1 carbon atom. Examples of amide organic solvents of Formula (III) include, but are not limited to, formamide, N,N-dimethylformamide, N,N-dimethylacetamide, N,N-diethylacetamide, N-methyl-2-pyrrolidone, and N-ethyl-2-pyrrolidone.

Specific examples of solvents which can be used include mono alcohols (e.g. C₂ to C₈ alcohols, including primary, secondary and tertiary alcohols), poly alcohols (e.g. ethylene glycol, propylene glycol, glycerine), diethylene glycol ethyl ether (Carbitol™), 1-methoxy-2-propanol, N-methyl pyrrolidone, acetonitrile, chlorobenzene, 1,4-dioxane, ethyl acetate, methyl ethyl ketone, tetrahydrofuran, toluene, xylene and mixtures thereof.

The following solvents are sometimes preferred: ethanol, 1-methoxy-2-propanol, N-methyl pyrrolidone, diethylene glycol ethyl ether, and mixtures thereof. In some situations, suitable solvents may also include low boiling alcohols (below 100° C.; like methanol, ethanol, propanol) and mixtures thereof or preferably the same solvent(s) described above.

The liquid is typically present in the following amounts: at least 20 or at least 25 or at least 30 wt. %; at most 70 or at most 65 or at most 60 wt. %; 20 to 70, or 25 to 65, or 30 to 60 wt. %;
wt. % with respect to the weight of the printing sol.

Coloring components which can be used include in particular components comprising ions of Fe, Mn, Er, Pr, Tb, Nd, Cr, Co, Mo. If present, these components are typically present as oxides in the sintered article.

The coloring ions are typically present in the following amounts: 0 or at least 0.01 or at least 0.02 wt. %; at most 2 or at most 1 or at most 0.8 wt. %; 0 to 2, or 0.01 to 1, or 0.02 to 0.8 wt. %; wt. % total coloring ions with respect to the weight of the printing sol.

The coloring components can be added as a separate component to the printing sol and/or can be part of the crystalline zirconia particles.

According to a further embodiment, the printing sol described in the present text comprises one or more inhibitor(s).

The nature and structure of the inhibitor(s) is not particularly limited, either, unless the desired result cannot be achieved.

An inhibitor may extend the shelf life of the printing sol, help prevent undesired side reactions, and adjust the polymerization process of the radiation curable component(s) present in the sol.

Adding one or more inhibitor(s) to the printing sol may further help to improving the accuracy or detail resolution of the surface of the ceramic article.

In particular it was found that adding inhibitor(s) to the printing sol described in the present text may help to enhance the resolution and accuracy of the SLA process by attenuating or avoiding unwanted scattering effects, as well as increase the shelf life of the printing sol.

The inhibitor(s) should be soluble in the solvent contained in the sol. Inhibitors which can be used often comprise a phenol moiety.

Specific examples of inhibitor(s) which can be used include: butylhydroxytoluol (lonol), p-methoxyphenol (MOP), hydroquinone monomethylether (MEHQ), 2,6-di-tert-butyl-4-methyl-phenol (BHT), phenothiazine, 2,2,6,6-tetramethyl-piperidine-1-oxyl radical (TEMPO) and mixtures thereof.

The inhibitor is typically present in the following amounts: 0 or at least 0.001 or at least 0.01 wt. %; at most 0.5 or at most 0.2 or at most 0.15 wt. %; 0 to 0.5 or 0.001 to 0.2 or 0.01 to 0.15 wt. %; wt. % with respect to the weight of the printing sol.

With respect to its physical properties the radiation-curable zirconia sol A can be characterized by the following features alone or in combination:
viscosity: less than 500 mPa*s at 23° C.;
being translucent in a wavelength range of 420 to 600 nm for a pathlength of 10 mm;
showing a transmission of at least 5% at a wavelength of 420 nm for a pathlength of 10 mm;
pH value: 1 to 6.

It was found that using a translucent printing sol can be beneficial for improving the accuracy or detail resolution of the surface of the ceramic article. Translucent printing sols show less scattering of light, which is used for polymerizing the radiation-curable components contained in the sol. The increased translucency allows for a shallower cure gradient, which may also allow for a more uniform cure across the entire structure to be obtained, as lower energy doses are required to cure through a translucent material.

A printing sol having a viscosity in the above range is beneficial e.g., as it can easily be processed through thin nozzles and tubes. There is no need for heating either the nozzles or tubes of the manufacturing unit or the manufacturing material itself. Low viscosity sols quickly self-level on a surface. Low viscosity sols also allow for simple removal of excess materials from internal channels or deep slots, such as in the case of a narrow wire slot on an orthodontic bracket. Higher viscosity sols combined with a green body of lower mechanical strength make the likelihood of fully realizing the small resolution of channels and dips rather low.

The printing sols typically have a viscosity that is Newtonian or nearly Newtonian like. That is, the viscosity is independent of shear rate or has only a slight dependence on shear rate.

The printing sol described in the present text can be obtained as follows:

A starting sol containing nano-sized particles is provided.

A precursor solution is typically prepared by combining a zirconium salt (e.g. acetate) solution and a solvent (e.g. water). A phase stabilizing agent (e.g. yttrium acetate) is added and dissolved in the precursor solution. Optionally, a coloring component (e.g. iron acetate) is also added to the precursor solution. The resulting composition is pumped e.g. through a hydrothermal reactor.

When subjected to hydrothermal treatment, the various dissolved salts undergo hydrolysis and condensation reactions to form zirconia-based particles. These reactions are often accompanied with the release of an acidic by-product (e.g. acetic acid).

Suitable hydrothermal reactors are described e.g. in U.S. Pat. No. 5,453,262 (Dawson et al.) and U.S. Pat. No. 5,652,192 (Matson et al.).

The content of tetragonal and/or cubic phase of the zirconia crystallites can be adjusted by varying the amount of phase stabilizing components added during the production method.

The starting sol is typically concentrated.

To obtain a more concentrated sol, at least a portion of the aqueous-based medium is removed from the zirconia-based sol.

According to one embodiment, the zirconia-based sol is subjected to dialysis or diafiltration.

The content of the crystalline nano-sized zirconia particles in the concentrated starting sol is typically in a range from 20 to 70 wt. %.

In some embodiments, the zirconia-based sol can be subjected to a solvent exchange process.

An organic liquid having a higher boiling point than water can be added to the effluent.

Examples of organic liquids that are suitable for use in a solvent exchange method include 1-methoxy-2-propanol, N-methyl pyrrolidone ordiethylene glycol ethyl ether. The water then can be removed by a method such as distillation, rotary evaporation, or oven drying.

A zirconia-based sol comprises zirconia-based particles dispersed and/or suspended (i.e., dispersed, suspended, or a combination thereof) in an aqueous/organic matrix.

To the starting sol the other components are added: the radiation-curable component(s), the photo-initiator(s), and optionally organic dye(s), inhibitor(s), soluble inorganic coloring agent(s), and other additive(s), if desired.

The preparation of the printing sol is typically conducted under safe light conditions to avoid an undesired early polymerization.

In a further step a radiation-curable zirconia sol Bₓ is applied on at least a portion of the outer surface of the zirconia gel article. The radiation-curable zirconia sol Bₓ is also referred to as coating sol.

The composition of the radiation-curable zirconia sol Bₓ can be same or different from the composition of radiation-curable zirconia sol A with respect to chemical composition and/or physical properties.

If the composition is different, the radiation-curable zirconia sol Bₓ differs from the composition of the radiation-curable zirconia sol A by the following features alone or in combination:
a) color or content of coloring components;
b) content of crystal phase stabilizer component;
c) viscosity;
d) wt. % of crystalline zirconia particles in the sol;
wherein a combination of a) and b), or a) and c), or b) and c), or b) and d) is sometimes preferred.

In some embodiments, the difference in viscosity between the sols is typically at least greater than a ratio of 1.2 or 1.5 or 2.

Applying an additive-manufacturing process for processing a radiation-curable chemical composition is generally known. Such a process is also often referred to as 3d-printing process.

Such a process typically comprises the following steps:
providing a layer of the construction material on a surface,
radiation curing those parts of the layer of construction material which will belong to the 3-dim article to be produced,
providing an additional layer of the construction material in contact with the radiation cured surface of the previous layer,
repeating the previous steps until a 3-dim article is obtained.

Such a process comprises the step of applying radiation to the surface of a radiation-curable material, wherein the radiation is applied only to those parts of the surface which will later form a part of the article to be produced.

Radiation can be applied by using e.g. a laser beam or by mask-image projection. Using a mask-image projection based stereolithography process (MIP-SL) is sometimes preferred, as it allows a more rapid manufacturing of the article.

A MIP-SL process can be described as follows:
i. A three-dimensional digital model of the article to be produced is provided.
ii. The three-dimensional digital model is sliced by a set of horizontal planes.
iii. Each thin slice is converted into a two-dimensional mask image.
iv. The mask image is then projected with the aid of a radiation source onto the surface of the radiation curable material being located in a building platform (e.g. having the shape of a vat).
v. The radiation curable material is only cured in those regions which are exposed.
vi. The building platform containing the radiation curable material or the layer of cured material is moved relative to the radiation source, wherein a new layer of radiation curable material is provided being in contact with the layer of the cured material produced in the previous step.
vii. Steps (iv) to (vi) are repeated until the desired article is formed.

Projecting the mask image on the radiation curable material can be done either top-down or bottom-up with respect to the orientation of the vat.

Using the bottom-up technique can be beneficial as less radiation curable material is needed.

It was found that the radiation-curable zirconia sols described in the present text are in particular useful for processing in a mask-image projection stereolithography process using the bottom-up projection technique.

Further details of such a processing step are described in U.S. Pat. No. 4,575,330 (Hull), U.S. Pat. No. 6,283,997 (Garg et al.) or U.S. Pat. No. 8,003,040 B2 (El-Siblani).

The processing of the radiation-curable zirconia sol can be done by using or applying the following parameters alone or in combination:
slice thickness of radiation-curable zirconia sol exposed to radiation: 0.001 to 0.500 mm or 0.01 to 0.4 mm;
energy dose per layer in the range of 5 mJ/cm² to 100 mJ/cm² or 8 mJ/cm² to 50 mJ/cm².

The application of the radiation-curable zirconia sol Bₓ can be done manually or automated using e.g., a robot.

Using an automated system may help to increase the throughput and facilitate the repeatability of the application step.

For automation purposes using e.g., a gantry robot design was found to be suitable. The radiation-curable zirconia sol Bₓ can be applied with the aid of a mechanical tool such as a brush, a spray nozzle or by using an inkjet-system. The radiation-curable zirconia sol Bₓ can be applied to the surface only once or repeatedly, if desired.

If desired, different radiation-curable zirconia sols Bₓ (B₁, B₂, B₃, B₄, etc.) can be applied.

Applying different radiation-curable zirconia sols Bxcan help to further customize the dental zirconia article e.g., with respect to color and/or translucency.

If desired, the radiation-curable zirconia sol Bₓ can also be applied via a top down stereolithography or volumetric additive-manufacturing process.

E.g. a top down continuous manufacturing device as described e.g. in US 2022/0380260 A1 (Shah et al.) can be used to create a gel body comprised of a precision print from a first sol (e.g., printing sol A), an individually-shaped interface between two regions, and a 3 dimensionally controlled coating of a second sol (e.g., coating sol Bₓ). Such a process may comprise the following steps:
a first gel body is created with a pre-defined reference orientation, wherein the first gel body is based on a first printable sol;
a second printable sol is placed in a manufacturing device comprising a printer vat with top down projection of light and elevator for moving the part into the sol;
the first gel body is processed in the manufacturing device in such an orientation that the region to receive the coating of the second sol is not undercut to the first region. (I.e. is accessible to curing from the top down projection of light. a
a second print is created on top of the first print by advancing the first gel body into the resin bath while projecting the appropriate light pattern to solidify the second printable sol into a gel.

The resulting multi-component gel may be processed into a fully dense ceramic using methods described elsewhere in this text.

Alternatively, a volumetric additive-manufacturing system may be used to build up multi-component gel bodies. Volumetric approaches can have advantages in their ability to print on top of nonplanar surfaces, require few supports as well as generating smooth surfaces. Useful systems have been described by Toombs et al (Science 376, Apr. 15, 2022) and Madrid Woolfe et al (Adv. Sci. 2022, 9, 2105144) and Regehly et al (Nature 2020, Vol 588 p 620-24) and Huffman et al. Multiphoton Imaging Methods in a Scattering and/orAbsorbing Medium, and Articles (US20220350127A1). Such a process may comprise the following steps:

A first gel body is created with a pre-defined reference orientation, wherein the first gel body is based on a first printable sol; this printer may be of the types described above, including a volumetric system.

A second printable sol is placed in a volumetric printer and the first gel body is arranged in a known orientation in the sol bath in the printer.

A second print is generated on the surface of the gel body while the light source(s) and printer vat are moved relative to one another and the appropriate light patterns are projected.

Alternatively, the sol composition in the volumetric printer is changed in a coordinated manner during a print by flowing a dopant into the bath while the appropriate light pattern is projected into the vat to render the desired restoration portions in the composition present at the time of light exposure.

The resulting multi-component gel may be processed into a fully dense ceramic article using methods described the present text or known to the skilled person. Additional sols, representing different characteristics may be printed on the article as well. Some additional characteristics include hypocalcifications, decalcifications, cracks, various stains, etc. One particular advantage of the additive manufacturing approach is that the presence or absence of coating can be locally controlled across the printable surface. Further, the thickness of the coating can be locally controlled from a coating on the order of a voxel thick to a coating on the order of mm. When designed carefully, the coated article from carefully selected sols can resemble natural dentin and enamel layers.

The curing of the radiation-curable zirconia sols is typically done by radiation. The wavelength of the radiation is typically in a range of 300 to 500 nm or 365 to 460 nm.

According to one embodiment the processing of the radiation-curable zirconia sol A and the radiation-curing of zirconia sol Bₓ is done at different radiation wavelengths.

E.g., the curing of radiation-curable zirconia sol A is done at a wavelength in the range of 365 to 420 nm, whereas the curing of radiation-curable zirconia sol Bₓ is done at a wavelength in the range of 420 to 460 nm. Thus, the wavelength used for curing the radiation-curable zirconia sol A can be shorter than the wavelength used for curing the radiation-curable zirconia sol Bₓ.

In multiphoton printing the wavelength is twice the wavelength required to activate the initiator. In Xolography one wavelength may be used to activate a second initiator.

Upconversion photopolymerization strategies (Sanders, et al Nature 2022 604 474-478) may also be employed to permit printing at longer wavelengths, where light scattering by nanoparticles and interference from coloring ions is minimized.

The process described in the present text also comprises the step of transforming the zirconia gel article into a zirconia aerogel article.

The zirconia aerogel article is formed by removing liquid from the zirconia gel article without excessive shrinkage (e.g., not greater than about 30 or 40 vol. %).

Water, if present, can be removed from the zirconia gel article via alcohol exchange to provide an at least partially de-watered gel. The zirconia gel article is then converted or transformed to a zirconia aerogel article by removing the alcohol, if present, from the partially de-watered zirconia gel article via e.g. super critical extraction.

The zirconia aerogel article can be characterized by the following features alone or in combination:
(a) comprising crystalline zirconia particles having an average primary particle size in a range of 2 nm to 50 nm or 2 nm to 30 nm or 2 to 20 nm;
(b) content of crystalline zirconia particles: at least 40 wt. %;
(c) having an organic content of at least 3 wt. % or within a range of 3 to 50 wt. %;
(d) length of two of the x, y, z dimensions: at least 3 or at least 5 or at least 8 mm;
(e) density: 0.5 to 1.5 g/cm³.

A combination of the features (a) and (b), or (a), (b) and (c), or (b), (d) and (e) can be preferred.

If applied, the supercritical extraction step can be characterized by at least one, more or all of the following features:
(a) temperature: 20 to 100° C. or 30 to 80° C. or 15 to 150° C.;
(b) pressure: 5 to 200 MPa or 10 to 100 MPa or 1 to 20 MPa or 5 to 15 MPa;
(c) duration: 2 to 175 h or 5 to 25 h or 1 to 5 h;
(d) extraction or drying medium: carbon dioxide in its supercritical stage.

A combination of features (a), (b) and (d) is sometimes preferred.

Supercritical extraction can remove all or most of the liquid in the printed gel article. In some embodiments, the aerogels contain some residual organic liquid. The residual liquid can be up to 6 wt. % based on the total weight of the aerogel article. For example, the aerogel article can contain up to 5 wt. %, up to 4 wt. %, up to 3 wt. %, up to 2 wt. %, or up to 1 wt. % organic liquid.

The removal of organic liquid results in the formation of pores within the dried structure. Preferably, the pores are sufficiently large to allow gases from the decomposition products of the polymeric material to escape without cracking the structure when the dried structure is further heated to burnout the organic material and to form a sintered article.

In a further step, the zirconia aerogel article is typically heat-treated.

Depending on the heat-treatment conditions the heat-treatment may yield a calcined dental zirconia article, a pre-sintered dental zirconia article, or a sintered dental zirconia article.

The heat treatment can be conducted in one or more steps and if desired at different physical locations.

For obtaining a calcined dental zirconia article, a heating step is conducted to remove organic residues which are still present in the zirconia article before final sintering. Removing organic residues before sintering reduces the risks of cracks during sintering.

The heating step is typically conducted at a temperature below 800° C. or below 700° C. or below 600° C. A typical temperature range is 400 to 800° C. or 500 to 700° C.

The heating step is typically conducted for a time needed to combust the organic components in the zirconia article. A typical time frame is from 5 to 100 h or from 10 to 50 h.

For obtaining a pre-sintered dental zirconia article the following conditions are typically applied alone or in combination:
temperature: 900 to 1,100° C. or 950 to 1,090° C. or 975 to 1,080° C.; atmosphere: air or inert gas (e.g. nitrogen, argon);
dwell time: 0 to 24 h, or 0.1 to 5 h;
duration: until a density of 40 to 60% of the density of the sintered material has been reached.

The dwell time (that is the time during which the aerogel article is kept at that temperature) is helpful as well to tune strength and/or hardness to the specific needs and may help to facilitate the removing of support structures which may have been created during the additive-manufacturing step.

For obtaining a sintered dental zirconia article the following conditions are typically applied:
temperature: 1,100 to 1,400° C. or 1,200 to 1,350° C.;
atmosphere: air or inert gas (e.g. nitrogen, argon);
duration: until the porosity of the zirconia article is less than 0.1 or less than 0.05 vol. %;
dwell time: 0.1 to 6 h or 0.2 to 5 h;
pressure: 500 to 1,500 hPa.

It is, however, also possible to provide a pre-sintered dental zirconia article first which is later sintered to a sintered dental zirconia article.

During the heating step the porous dental ceramic article is sintered to its final shape, thereby undergoing changes with regard to dimension, density, hardness, bending strength and/or grain size.

The heating temperature and dwell time (that is, the time period during which a particular temperature is kept) are typically correlated. A higher temperature typically requires only a short dwell time.

The process described in the present text may comprise additional steps, if desired.

According to one embodiment, the radiation-curable sol Bₓ is applied on portions of the outer and inner surface of the zirconia gel article.

This embodiment may contribute to achieving glossy surfaces on both sides of the dental article. Alternatively, a glossy surface may be obtained on the outer surface while the inner surface retains a more matte finish.

According to one embodiment, at least two different radiation-curable sols B_{1,2} are applied on portions of the outer and/or inner surface of the zirconia gel article.

If differently colored zirconia sols are used, differently colored surfaces can be realized.

This allows the provision of customized dental articles adapted to the individual situation in the mouth of a patient.

Applying a more opaque zirconia sol Bₓ on the inner surface of the dental article can be advantageous to mask a dark surface of a tooth stump without affecting the depth of reflections on the outer surface of the dental article.

According to one embodiment, a radiation-curable sol B₁ is applied on portions of the outer surface of the zirconia gel article and a radiation-curable sol B₂ is applied on portions of the inner surface of the zirconia gel article.

The process described in the present text may also comprise the following steps alone or in combination:
(a) cleaning step;
(b) drying step;
(c) post-curing step;
(d) ion-exchange step;
(e) step of removing support structures, if present, e.g. from the sintered zirconia ceramic article or at an earlier stage.

A combination of the following steps is sometimes preferred: (a) and (c); (a), (c) and (e); (a), (b), (c) and (e).

After processing the radiation-curable zirconia sol A, the obtained zirconia gel article is typically removed from the device which has been used for conducting the additive manufacturing process.

If desired, the surface of the zirconia gel article is cleaned, e.g. by rinsing the zirconia gel article with a liquid or soaking in a liquid.

Suitable liquids preferably include mixtures thereof or the same liquids(s) used in the sols described in the present text.

Suitable liquids include either low boiling alcohols as described in the present text (e.g. an alcohol having a boiling point below 100° C.; like methanol, ethanol, propanol) and mixtures thereof or high boiling solvents as described in the present text, preferably the same liquids(s) being present in the sol, e.g. diethylene glycol ethyl ether.

Alternatively, cleaning may consist of simply allowing the uncured sol on the part to settle to a desired level. This process can be facilitated by spinning the part to allow centrifugal forces to remove excess material while rendering a smooth and typically glossy surface, which is typically cured in place with light exposure.

If desired, a drying step can be conducted for removing any organic liquids or water, if present, from the zirconia gel article, at least partially.

Such a drying step typically takes place before a supercritical extraction step is conducted.

Drying is typically done at a temperature in the range of 20 to 200° C. under ambient conditions.

If desired, the zirconia gel article can be post-cured by applying radiation or heat.

Such a step helps to improve the stability of the zirconia gel article by further increasing the degree of polymerization.

If present, the post-curing step can be characterized by the following features alone or in combination:
applying radiation with wavelength of 200 to 500 nm or 350 to 460 nm;
applying a heating step with a temperature below the temperature at which drying will occur or which is used for de-bindering or calcining; e.g. 30 to 110° C. or 40 to 80° C.

A post-curing step is typically conducted after the step of radiation-curing of the radiation-curable zirconia sol Bₓ.

The pre-sintered article optionally can be soaked in a basic solution such as an aqueous solution of ammonium hydroxide. Soaking can be effective to remove undesirable ionic species such as sulfate ions because of the porous nature of the articles at this stage of the process.

After soaking, the article is removed from the solution and washed thoroughly with water. The article can be soaked in water for any desired period of time such as at least 30 min, at least 1 hour, at least 2 hours, or at least 4 hours. The soaking in water can be repeated several times, if desired, by replacing the water with fresh water.

After soaking, the article is typically dried in an oven to remove the water. For example, the article can be dried by heating in an oven set at a temperature equal to at least 80° C., at least 90° C., or at least 100° C. For example, the temperature can be in a range of 80° C. to 150° C., 90° C. to 150° C., or 90° C. to 125° C. for at least 30 min, at least 60 min, or at least 120 min.

An ion-exchange step can typically be characterized by the following features alone or in combination: duration: 5 to 24 h; medium: aqueous ammonium hydroxide liquid (e.g., concentration 1N); temperature: 20 to 25° C.

Depending on the additive-manufacturing method used, the obtained 3d-printed article may comprise support structure.

If present, these support structures need to be removed before the fixing the dental article to a tooth in the mouth of a patient.

The support structure can be removed at any suitable time during the process. The support structure can be removed after any of the following steps: post-curing step, step of transforming the zirconia gel article to a zirconia aerogel article, ion-exchange step, heat-treating step (e.g. calcining, pre-sintering or sintering step).

The removal can be accomplished manually or by an automated process. Typically, a cutting tool is used.

The invention is also directed to a dental article obtainable or obtained by the process described in the present text. Such a dental article has an outer and an inner surface.

The dental article may have the shape of a dental crown, bridge, inlay, onlay, veneer, bracket, buccal tube, cleat or button.

The dental article can be a pre-sintered dental zirconia article or a sintered dental zirconia article.

The pre-sintered dental zirconia article can be characterized by the following features alone or in combination:
density: 2.5 to 3.5 g/cm³;
showing a N₂ adsorption and desorption of isotherm type IV according to IUPAC classification and a hysteresis loop;
showing a N₂ adsorption and desorption isotherm of type IV with a hysteresis loop of type H1 according to IUPAC classification;
showing a N₂ adsorption and desorption isotherm of type IV with a hysteresis loop of type H1 according to IUPAC classification in a p/p0 range of 0.70 to 0.99;
BET-surface: 15 to 100 m²/g or 16 to 60 m²/g.

The material of the pre-sintered dental zirconia article can be characterized by the following features alone or in combination:
ZrO₂ content: 70 to 98 mol %;
HfO₂ content: 0 to 2 mol %;
Y₂O₃ content: 2 to 8 mol %;
Al₂O₃ content: 0 to 0.5 mol %;
coloring components: 0 to 1 mol % comprising oxides selected from Fe, Mn, Er, Pr, Tb, Nd, Cr, Co, Mo and mixtures thereof,
   mol % with respect to the pre-sintered dental zirconia article.

According to a further embodiment, the pre-sintered dental zirconia article has a composition being characterized by the following features:
ZrO₂ content: 90 to 98 mol %,
HfO₂ content: 0 to 2 mol %,
Y₂O₃ content: 3 to 6 mol %,
Al₂O₃ content: 0 to 0.1 mol %,
coloring components: 0.01 to 0.5 mol % comprising oxides selected from Fe, Mn, Er, Pr, Tb, Nd, Cr, Co, Mo and mixtures thereof,
   mol % with respect to the pre-sintered dental zirconia article.

It was found that a higher Y₂O₃ content typically leads to an increase of the cubic crystal phase in the zirconia ceramic material after sintering the material to final density. A higher content of the cubic crystal phase may contribute to a better translucency.

The dental article in its sintered state may be characterized by the following features alone or in combination:
(a) thickness between outer and inner surface at at least one section: 0.3 to 0.5 mm;
(b) surface roughness of the surface area to which zirconia sol Bₓ has been applied: less than 0.4 μm;
(c) gloss of the surface area to which zirconia sol Bₓ has been applied: at least 50 GU, or in a range of 50 to 200 GU or 80 to 180 GU;
(d) opalescence: at least 10.

A combination of the features (a) and (b), or (a) and (c), or (a), (b) and (d), or (a), (b) and (c) is sometimes preferred.

Depending on the chemical composition and related crystal structure, a density of more than 5.80 g/cm³ or more than 5.90 g/cm³ or more than 6.00 g/cm³ or more than 6.05 g/cm³ is an indication that the dental article is close to a state of being fully sintered or has been fully sintered and thus is essentially fully dense.

The dental article typically has a porosity of less than 0.1 vol. % or less than 0.05 vol. %.

The surface of the dental article shows a superior reflectance or gloss, which essentially matches with the reflectance of the surface of natural teeth.

The process described in the present text allows the manufacturing of dental articles with thin walls.

The opalescence of the dental article is in a range which also matches with the opalescence of natural teeth.

Due to the application of radiation-curable zirconia sol Bₓ on at least portions of the outer surface of the zirconia gel article, the roughness of the surface of dental article after the heat-treating step can be reduced to a range which is acceptable to the patient.

The surface roughness is typically <0.4 μm or <0.3 μm or <0.2 μm.

The volume of the zirconia gel article is typically larger than the volume of the zirconia aerogel article by a factor of at least 1.1, or at least 1.2 or at least 1.3.

The volume of the zirconia aerogel article is typically larger than the volume of the zirconia sintered article by a factor of at least 2, or at least 3 or at least 4.

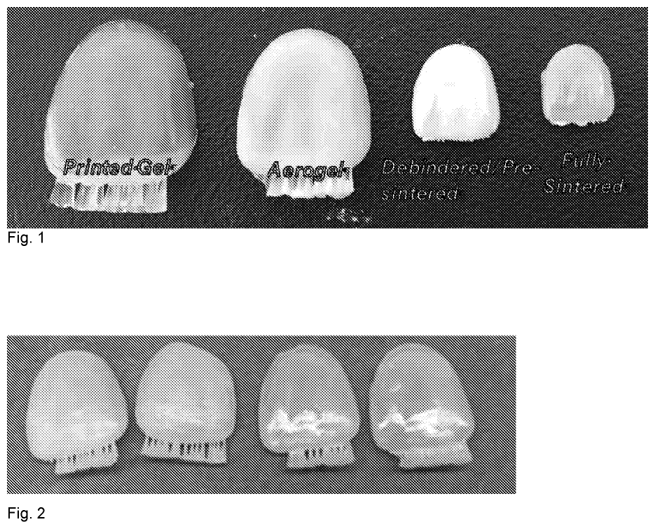

Thus, during the whole process, the volume of the dental article is reduced. This is also shown in FIG. 1.

The chemical composition of the sintered zirconia article is essentially the same as the chemical composition of the pre-sintered zirconia article. The content of oxide components will not change.

More specific embodiments of the process and the dental article described in the present text are given below:

### Embodiment 1

The process may comprise, essentially consist of, or consist of the following steps:
(a) providing a radiation-curable zirconia sol A comprising nano-sized crystalline zirconia particles, crystal phase stabilizer component, liquid, a radiation-curable component, photo-initiator, optionally coloring component(s) and optionally inhibitor component(s), as described in the present text, wherein the crystal phase stabilizer component is contained in the crystalline zirconia particles and the optional coloring component can be contained in the crystalline zirconia particles or be present as a separate component,
(b) processing the radiation-curable zirconia sol A as construction material in an additive manufacturing process to obtain a 3-dim zirconia gel article,
(c) optionally cleaning the surface of the 3-dim zirconia gel, in particular for the purpose of removing residues of non-reacted sol,
(d) applying radiation-curable zirconia sol Bₓ on at least a portion of the outer surface of the 3-dim zirconia gel article,
(e) post-curing (e.g. by heating or light-curing) the 3-dim zirconia gel article to a temperature in the range of 35 to 80° C. or by additional light curing, in particular for the purpose of increasing the stability of the zirconia gel article,
(f) optionally soaking the 3-dim zirconia gel article with another liquid (e.g. diethylene glycol ethyl ether or ethanol),
(g) transforming the 3-dim zirconia gel article to a 3-dim zirconia aerogel article, preferably by applying a supercritical drying step, in particular for the purpose of removing liquid,
(h) heating the 3-dim zirconia aerogel article to a temperature in the range of 400 to 800° C., in particular for the purpose of removing residual organic components and to further increasing the stability,
(i) heating the 3-dim article of the previous step to a temperature in the range of 800 to 1,100° C., in particular for the purpose of creating a 3-dim pre-sintered body having a porous structure,
(j) optionally conducting an ion-exchange step,
(k) applying a sintering step to obtain a 3-dim sintered zirconia ceramic article,
(l) optionally removing support structures remaining from the processing step (b) from the article, if support structures are present, wherein step (I) can be conducted after any of steps l, (g), (i) or (k).

### Embodiment 2

A dental ceramic zirconia article, in particular obtainable or obtained by a process described in the present text, the dental ceramic zirconia article being characterized by the following properties:
thickness between outer and inner surface at at least one section: 0.5 to 0.3 mm,
surface roughness of the surface area to which zirconia sol Bₓ has been applied: <0.4 μm,
gloss: at least 50 GU,
opalescence: at least 10,
ZrO₂ content: 90 to 98 mol %,
HfO₂ content: 0 to 2 mol %,
Y₂O₃ content: 3 to 6 mol %,
Al₂O₃ content: 0 to 0.1 mol %,
   mol. % with respect to the dental ceramic zirconia article.

The process described in the present text does typically not comprise a step of applying a glazing layer after the sintering step.

In particular, the dental zirconia article described in the present text is not coated with a material other than the material used for producing the dental zirconia article. In this respect the dental zirconia article can be regarded as a monolithic dental zirconia article.

The radiation-curable zirconia sols described in the present text are typically stored in suitable packaging material before use to avoid an undesired premature curing of the sol.

Suitable packaging materials include vessels, bottles, cartridges, containers or foil bags, typically with a volume in the range of 10 to 10,000 ml.

The dental zirconia article described in the present text is particularly useful for restoring a defect tooth in the mouth of a patient.

The defect tooth may be a molar or a front tooth, wherein the restoration of a front tooth is sometimes preferred.

Thus, the dental zirconia article is for use in a process of treating a defect tooth in the mouth of a patient.

Such a process typically comprises the step of fixing the inner surface of the dental zirconia article to the surface of a tooth to be treated.

The fixing can be accomplished by using a dental cement, if desired in combination with a dental adhesive and/or dental primer.

Dental adhesives directly interact with the enamel or dentin surface of a tooth. Dental adhesives are typically one-part compositions, are radiation-curable and comprise ethylenically unsaturated component(s) with acidic moiety, ethylenically unsaturated component(s) without acidic moiety, water, sensitizing agent(s), reducing agent(s) and additive(s).

Examples of dental adhesives are described in US 2020/0069532 A1 (Thalacker et al.) and US 2017/0065495 A1 (Eckert et al.). Dental adhesives are also commercially available, e.g., 3M™ Scotchbond™ Universal or 3M™ Scotchbond™ Universal Plus (3M Oral Care).

Suitable dental primers are described in U.S. Pat. No. 6,126,922 (Rozzi et al.) and WO 00/69393 A1 (3M). Dental primers are also commercially available, e.g., 3M™ Transbond™ XT Primer (3M Oral Care).

Dental cements which can be added to the kit include in particular self-adhesive resin cements, which contain an acidic polymerizable component (e.g., a (meth)acrylate component bearing a phosphoric or carboxylic acid moiety), polymerizable components without an acidic moiety, an initiator system and filler.

Suitable dental cements are also commercially available, such as RelyX™ Unicem 2, RelyX™ Universal, RelyX™ Veneer or RelyX™ Luting Plus (3M Oral Care).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

### EXAMPLES

Objects and advantages of this disclosure are further illustrated by the following examples, but the particular materials and amounts thereof recited in these examples, as well as other conditions and details, should not be construed to unduly limit this disclosure.

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1,013 mbar at maritime level).

### Methods

### Method for Crystalline Structure and Size (XRD Analysis)

Dried zirconia samples were ground by hand using an agate mortar and pestle. A liberal amount of the sample was applied by spatula to a glass microscope slide on which a section of double-sided adhesive tape had been adhered. The sample was pressed into the adhesive on the tape by forcing the sample against the adhesive with the spatula blade. Excess sample was removed by scraping the sample area with the edge of the spatula blade, leaving a thin layer of particles adhered to the adhesive. Loosely adhered materials remaining after the scraping were removed by forcefully tapping the microscope slide against a hard surface. In a similar manner, corundum (Linde 1.0 μm alumina polishing powder, Lot Number C062, Union Carbide, Indianapolis, IN) was prepared and used to calibrate the X-ray diffractometer for instrumental broadening.

X-ray diffraction scans were obtained using a Philips vertical diffractometer having a reflection geometry, copper Kₐ radiation, and a proportional detector registry of the scattered radiation. The diffractometer was fitted with variable incident beam slits, fixed diffracted beam slits, and a graphite diffracted beam monochromator. The survey scan was recorded from 25 to 55 degrees two theta (2q) using a step size of 0.04 degrees and a dwell time of 8 seconds. X-ray generator settings of 45 kV and 35 mA were used. Data for the corundum standard was collected on three separate areas of several individual corundum mounts. Likewise, data was collected on three separate areas of the thin layer sample mount.

The observed diffraction peaks were identified by comparison to reference diffraction patterns contained within the International Center for Diffraction Data (ICDD) powder diffraction database (sets 1-47, ICDD, Newton Square, PA, USA). The diffraction peaks for the samples were attributed to either cubic/tetragonal (C/T) or monoclinic (M) forms of zirconia. For zirconia-based particles, the (111) peak for the cubic phase and (101) peak for the tetragonal phase could not be separated so these phases were reported together. The amounts of each zirconia form were evaluated on a relative basis and the form of zirconia having the most intense diffraction peak was assigned the relative intensity value of 100. The strongest line of the remaining crystalline zirconia form was scaled relative to the most intense line and given a value between 1 and 100.

Peak widths for the observed diffraction maxima due to corundum were measured by profile fitting. The relationship between mean corundum peak widths and corundum peak position (2q) was determined by fitting a polynomial to these data to produce a continuous function used to evaluate the instrumental breadth at any peak position within the corundum testing range. Peak widths for the observed diffraction maxima due to zirconia were measured by profile fitting the observed diffraction peaks. The following peak widths were evaluated depending on the zirconia phase found to be present:
Cubic/Tetragonal (C/T): (1 1 1)
Monoclinic (M): (−1 1 1), and (1 1 1)

A Pearson VII peak shape model with Kₐ₁ and Kₐ₂ wavelength components and linear background model were used for all measurements. Widths were calculated as the peak full width at half maximum (FWHM) having units of degrees. The profile fitting was accomplished by use of the capabilities of the JADE diffraction software suite. Sample peak widths were evaluated for the three separate data collections obtained for the same thin layer sample mount.

Sample peaks were corrected for instrumental broadening by interpolation of instrumental breadth values from corundum instrument calibration and corrected peak widths converted to units of radians. The Scherrer equation was used to calculate the primary crystal size.

$$Crystallite Size ( D ) = K l / b ( cos q )$$

In the Scherrer equation, K is the form factor (here 0.9), l is the wavelength (1.540598 Å), b is the calculated peak width after correction for instrumental broadening (in radians), and q equals half the peak position (scattering angle). b is equal to [calculated peak FWHM−instrumental breadth] (converted to radians) where FWHM is full width at half maximum. The cubic/tetragonal (C/T) mean crystallite size was measured as the average of three measurements using (1 1 1) peak. That is,

$$C / T mean crystallite size = [ {D ( 111 )}_{area 1} + {D ( 111 )}_{area 2} + {D ( 111 )}_{area 3} ] / 3 .$$

### Method for Photon Correlation Spectroscopy (PCS)

Particle size measurements were made using a light scattering particle sizer equipped with a red laser having a 633 nm wavelength of light (obtained under the trade designation “ZETA SIZER—Nano Series, Model ZEN3600” from Malvern Instruments Inc., Westborough, MA). Each sample was analyzed in a one centimeter square polystyrene sample cuvette. The sample cuvette was filled with about 1 gram of deionized water, and then a few drops (about 0.1 gram) of the zirconia-based sol were added. The composition (e.g., sample) within each sample cuvette was mixed by drawing the composition into a clean pipette and discharging the composition back into the sample cuvette several times. The sample cuvette was then placed in the instrument and equilibrated at 25° C. The instrument parameters were set as follows: dispersant refractive index 1.330, dispersant viscosity 0.8872 MPa-second, material refractive index 2.10, and material absorption value 0.10 units. The automatic size-measurement procedure was then run. The instrument automatically adjusted the laser-beam position and attenuator setting to obtain the best measurement of particle size.

The light scattering particle sizer illuminated the sample with a laser and analyzed the intensity fluctuations of the light scattered from the particles at an angle of 173 degrees. The method of Photon Correlation Spectroscopy (PCS) was used by the instrument to calculate the particle size. PCS uses the fluctuating light intensity to measure Brownian motion of the particles in the liquid. The particle size is then calculated to be the diameter of sphere that moves at the measured speed.

The intensity of the light scattered by the particle is proportional to the sixth power of the particle diameter. The Z-average size or cumulant mean is a mean calculated from the intensity distribution and the calculation is based on assumptions that the particles are mono-modal, mono-disperse, and spherical. Related functions calculated from the fluctuating light intensity are the Intensity Distribution and its mean. The mean of the Intensity Distribution is calculated based on the assumption that the particles are spherical. Both the Z-average size and the Intensity Distribution mean are more sensitive to larger particles than smaller ones.

The Volume Distribution gives the percentage of the total volume of particles corresponding to particles in a given size range. The volume-average size is the size of a particle that corresponds to the mean of the Volume Distribution. Since the volume of a particle is proportional to the third power of the diameter, this distribution is less sensitive to larger particles than the Z-average size. Thus, the volume-average will typically be a smaller value than the Z-average size.

### Method for Measuring Oxide Content

The oxide content was measured via thermal gravimetric analysis (obtained under the trade designation “TGA Q500” from TA Instruments, New Castle, DE, USA). The sample (about 50 mg) was loaded into the TGA and the temperature was taken to 900° C. in air. The oxide content of the sample was equal to the residual weight after heating to 900° C.

### Method for Measuring Archimedes Density

The density of the sintered material was measured by the Archimedes technique. The measurements were made on a precision balance (identified as “XSE204” from Mettler-Toledo, LLC, Columbus, OH, USA) using a density determination kit (identified as “Density Determination Kit for Excellence XP/XS Analytical Balances” from Mettler-Toledo, LLC, Columbus, OH, USA). The sample was first weighed in air (A), then immersed in water and weighed (B). The water was distilled and deionized. Three drops of a wetting agent (obtained under trade designation “PERVITRO 75%” from Mettler-Toledo, LLC, Columbus, OH, USA) was added to 250 ml of water. The density was calculated using the balance density function, which used the formula ρ=(A/(A−B)) (ρ₀−ρ_{L})+ρ_{L}, where ρ₀ is the density of water and ρ_{L} is the density of air (0.0012 g/cm³). The relative density can be calculated by reference to the theoretical density (ρₜ) of the material, ρ_{ref}=(ρ/ρₜ)*100.

### Method for Measuring Opacity of Ceramic Articles

The opacity of a ceramic article was evaluated with the following procedure. After sintering, the dimensions of the printed ceramic test piece were approximately 1 mm±0.03 mm thick×13 mm×13 mm. The parallel large faces of the sample were polished to a surface finish of 15 micron grade diamond lapping film (668X Diamond Lapping Film PSA, 3M, St. Paul, MN) on a Beta, Grinder-Polisher (Buehler, Lake Bluff, IL), operating at 100 rpm and lubricated with water. The polished sample was measured with a spectrophotometer (X-Rite Color i7, Grand Rapids, MI, USA) in reflectance mode. Translucency (T) was determined according to T=1-RB/RW where RB is the reflectance through a ceramic sample on a black substrate and RW is the reflectance through the same sample on a white substrate. Higher values of translucency are indicative of greater transmission of light, and less opacity. Opacity (O) was determined according to O=100−T.

### Method for Surface Texture Analysis

The surface texture of samples was measured via laser confocal microscopy on a Keyence VK-X210 from Keyence Corporation, Osaka, Japan. Similar locations on each sample were located and imaged at 10× and 50× magnification, before taking a surface texture image with the scanning laser. Various numerical statistics were generated by included software, including R^{a}, average surface roughness, based on measurements averaged across 10 parallel horizontal lines 287 microns long, clustered around the center of the region imaged.

### Method for Measuring Viscosity

If desired, viscosity can be measured using a Physica MCR 301 Rheometer (Anton Paar, Graz, Austria) with a plate/plate geometry under controlled shear rate at 23° C. The diameter is 15 mm, the separation gap between the plates 0.5 mm. The shear rate is ramped from 1,000 s⁻¹ to 0.001 s⁻¹.

### Method for Measuring pH Value

If desired, the pH value of can be determined as follows: 1.0 g of a component (e.g. filler) is dispersed in 10 ml de-ionized water and stirred for about 5 min. A calibrated pH electrode is dipped into the suspension and the pH value is determined during stirring.

### Method for Measuring Elemental Composition

If desired, the elemental composition can be determined by X-ray fluorescence spectrometry (XRF), e.g. with the ZSX Primus II from Rigaku, Japan. This method is especially suited for the analysis of solids, e.g. zirconia ceramics or glass materials.

### Method for Measuring N₂ Sorption Isotherms, BET Surface Area, Pore Volume, Average Connected Pore Diameter

The samples are run on a QUANTACHROME AUTOSORB-1 BET Analyzer” (Quantachrome Instruments, Boynton Beach, FL). The samples are weighed and outgassed at 200° C. fortwo days then subjected to a N₂ sorption process with an appropriate number and distribution of measurement points, e.g. 55 adsorb points and 20 desorb points from a ρ/ρ₀ range 1×10⁶ to 1 and back to 0.05 giving full isotherms. The specific surface area S is calculated by the BET method (Details regarding calculation see Autosorb-1 Operating Manual Ver. 1.51 IV. Theory and Discussion; Quantachrome Instruments, Inc.). The total pore volume V_{liq} is derived from the amount of vapor adsorbed at a relative pressure close to unity (ρ/ρ₀ closest to 1), by assuming that the pores are then filled with liquid adsorbate (Details regarding calculation see Autosorb-1 Operating Manual Ver. 1.51 IV. Theory and Discussion; Quantachrome Instruments, Inc.). The average pore diameter (d) is calculated from the surface area (S) and the total pore volume

$$( {V}_{liq} ) \text{:} d = \frac{4 Vliq}{S} .$$

### Method for Measuring Reflectance/Gloss

The reflectance/gloss of the surface of an article can be determined using an instrument known as a Gloss Meter to measure Gloss Units (GU). Here, gloss samples are run on a Rhopoint Novo-Curve Gloss Meter (Rhopoint Americas, Troy, MI), calibrated at 60 degrees with a 93.7 GU and 0 GU standard. The procedure to measure a sample is as follows. Clean sample surface with isopropyl alcohol and wipe dry. Place sample with surface of interest on device aperture. Cover with black container (here a black plastic jar) to eliminate ambient light. Measure 60 degree specular reflection on device.

### Method for Measuring Opalescence

An article is considered opalescent, if the calculated opalescent value OP meets the following condition:

$$OP = {[ {( CIE {a}_{T}^{*} - CIE {a}_{R}^{*} )}^{2} + {( CIE {b}_{T}^{*} - CIE {b}_{R}^{*} )}^{2} ]}^{1 / 2} = at least 10 , or 10 to 40 or 15 to 40 ,$$

wherein
(CIEa_{T}*−CIEa_{R}*) is the difference between transmission and reflectance modes in red-green coordinate a*, and
(CIEb_{T}*−CIEb_{R}*) is the difference between transmission and reflectance modes in yellow-blue color coordinate b*.

The OP value can be determined with a sphere benchtop spectral photometer Color i7800 (x-rite, Michigan USA), applying the formula described above using samples of 1.0 mm in height and 15 mm in diameter.

| | Description |
|---|---|
| Zirconium acetate | An aqueous solution of zirconium acetate containing nominally |
| | 16.3 wt. % zirconium obtained from Magnesium Elektron, Inc. |
| | (Flemington, NJ, USA). The aqueous solution was exposed to an |
| | ion exchange resin (obtained under the trade designation |
| | “AMBERLYTE IR 120” from Rohm and Haas Company |
| | (Philadelphia, PA, USA) before use (oxide content 21.55 wt. %). |
| Yttrium acetate | Yttrium (III) acetate tetrahydrate obtained from Molycorp Inc. |
| | (Mountain Pass, CA) (oxide content 34.65 wt. %). |
| Iron acetate | Iron (II) acetate anhydrous obtained from Alfa Aesar (Ward Hill, |
| | MA, USA) (oxide content 40.84%). |
| Erbium acetate | Erbium (III) acetate tetrahydrate 99% (REO) obtained from Alfa |
| | Aesar (Ward Hill, MA, USA) (oxide content 46.48%). |
| DI water | De-ionized water. |
| Diethylene glycol | Diethylene glycol monoethyl ether obtained from Alfa Aesar (Ward |
| monoethyl ether | Hill, MA, USA). |
| “OMNIRAD 819” | UV/Visible photoinitiator obtained from IGM Resins (Waalwijk, The |
| | Netherlands) under the trade designation “OMNIRAD 819”. |
| MEEAA | 2-[2-(2-Methoxyethoxy)ethoxy]acetic acid obtained from Sigma- |
| | Aldrich (St. Louis, MO, USA). |
| Acrylic acid | Acrylic acid obtained from Alfa Aesar (Ward Hill, MA, USA). |
| HEA | 2-Hydroxyethyl acrylate obtained from Alfa Aesar (Ward Hill, MA, |
| | USA). |
| “SR351 H” | Trimethylolpropane triacrylate obtained from Sartomer USA |
| | (Exton, PA, USA) under the trade designation “SR351 H”. |
| “CN975” | Hexafunctional urethane acrylate obtained from Sartomer USA |
| | (Exton, PA, USA) under the trade designation “CN975”. |
| “CN9031” | Aliphatic Urethane Acrylate Oligomer obtained from Sartomer USA |
| | (Exton, PA, USA) under the trade designation “CN9031” |
| BHT | 2,6-Di-tert-butyl-4-methyl-phenol (Butylated hydroxytoluene) |
| | obtained from Fluka Analytical (St. Louis, MO, USA). |
| Isatin | Isatin 98%, ACROS Organics AC151491000 obtained from Fisher |
| | Scientific (Waltham, MA) |
| Ammonium | Ammonium Hydroxide (assay 28-30 wt. % as NH₃) obtained from |
| Hydroxide | EMD Chemicals Inc. (Gibbstown, NJ, USA). |

### Preparation Methods

### Sol Batch Preparation

Zirconia-based sols Sol-I(a)-Sol-V(a) were prepared as described in WO 2016/191534 A1 (Examples Section—Processing: Preparation of Sol-S1) except that the feed composition was varied. The target compositions for Sol-I(a)-Sol-V(a) are listed in Table 1, below.

TABLE 1

| | Mole % | Mole % | Mole % | Mole % |
|---|---|---|---|---|
| Sol | ZrO₂ | Y₂O₃ | Er₂O₃ | Fe₂O₃ |
| Sol-I(a) | 95.0 | 5.0 | — | — |
| Sol-II(a) | 95.0 | 5.0 | — | — |
| Sol-III(a) | 92.0 | 8.0 | — | — |
| Sol-IV(a) | 96.8 | 3.0 | — | 0.2 |
| Sol-V(a) | 93.8 | 5.0 | 1.2 | — |

The properties of Sol-I(a)-Sol-V(a) were determined using the methods described above. Table 2, below, summarizes the crystallite size from XRD analysis as well as the PCS data, including the volume-average size and the Z-average size.

TABLE 2

| | |
|---|---|
| XRD | PCS |

| | C/T size | Volume-Average Size | Z-Average Size |
|---|---|---|---|
| Sol | (nm) | (nm) | (nm) |
| Sol-I(a) | 5.9 | 11.03 | 16.67 |
| Sol-II(a) | 5.5 | 10.38 | 18.40 |
| Sol-III(a) | 5.4 | 12.11 | 19.05 |
| Sol-IV(a) | 6.7 | 9.85 | 21.27 |
| Sol-V(a) | 5.5 | 10.56 | 18.76 |

Sol-I(a)-Sol-V(a) were further processed to increase their concentration and/or remove acetic acid (AcOH). A combination of one or more of ultrafiltration, diafiltration and distillation were used. The diafiltration and ultrafiltration were performed using a membrane cartridge (obtained under the trade designation “M21S-100-01P” from Spectrum Laboratories Inc., Rancho Dominguez, CA). Distillation was performed using rotary evaporation.

Diethylene glycol monoethyl ether-based sols, Sol-I(b)-Sol-V(b), were prepared from Sol-I(a)-Sol-V(a) by adding 2-[2-(2-methoxyethoxy)ethoxy]acetic acid (MEEAA) (3.5-7.2 wt. % with respect to the grams of oxide in the sol) as well as the appropriate amount of diethylene glycol monoethyl ether (adjusted to the intended final oxide concentration in the sol, e.g., 55 wt. %) and concentrating each sol via rotary evaporation. The resulting weight percents of oxide, acetic acid (AcOH), and 2-[2-(2-methoxyethoxy)ethoxy]acetic acid (MEEAA) in each sol are given Table 3.

TABLE 3

| | wt. % | wt. % | wt. % |
|---|---|---|---|
| Sol | oxide | AcOH | MEEAA |
| Sol-I(b) | 50.22% | 6.74% | 2.68% |
| Sol-II(b) | 55.66% | 7.55% | 2.98% |
| Sol-III(b) | 54.05% | 6.78% | 3.85% |
| Sol-IV(b) | 55.60% | 5.70% | 1.98% |
| Sol-V(b) | 47.96% | 5.92% | 2.56% |

To prepare precursor-sol SPr1, 13.34 grams of Sol-IV(b) and 9.56 grams of Sol-V(b) were charged to a vial and combined with 2-[2-(2-methoxyethoxy)ethoxy]acetic acid (MEEAA) (0.024 gram), acrylic acid (1.30 grams), and diethylene glycol monoethyl ether (0.81 gram).

To prepare precursor-sol SPr2, 1096.6 grams of Sol-I(b) were charged to a 1-L bottle and combined with acrylic acid (59.53 grams) and diethylene glycol monoethyl ether (3.03 grams).

To prepare precursor-sol SPr3, 8.85 grams of Sol-III(b), 8.74 grams of Sol-IV(b), and 4.93 grams of Sol-V(b) were charged to a vial and combined with acrylic acid (1.30 grams) and diethylene glycol monoethyl ether (1.245 grams).

To prepare precursor-sol SPr4, 1303.4 grams of Sol-II(b) were charged to a 1-L bottle and combined with acrylic acid (77.66 grams) and diethylene glycol monoethyl ether (125.75 grams).

### Printing Sol Preparation

### Preparation of Printing Sol PSol1

To prepare printing sol PSol1, a portion of precursor-sol SPr2 (500 grams) was charged to a 1-liter bottle and combined with diethylene glycol monoethyl ether (33.5 grams), hydroxyethyl acrylate (HEA) (2.2 grams), trimethylolpropane triacrylate (“SR351H”) (47.1 grams), and two different urethane acrylates: CN975 (15.7 grams) and CN9031 (7.8 grams). Prior to printing, OMNIRAD 819 (0.1 wt. % with respect to the weight of the sol), butylated hydroxytoluene (BHT) (0.1 wt. % with respect to the weight of the sol), and isatin (0.05 wt % with respect to the weight of the sol) were dissolved in the sol. The printing sol was then passed through a 1-μm filter.

### Preparation of Printing Sol PSol2

To prepare printing sol PSol2, a portion of precursor-sol SPr4 (500 grams) was charged to a 1-liter bottle and combined with diethylene glycol monoethyl ether (33.5 grams), hydroxyethyl acrylate (HEA) (2.2 grams), trimethylolpropane triacrylate (“SR351H”) (47.1 grams), and two different urethane acrylates: CN975 (15.7 grams) and CN9031 (7.8 grams). Prior to printing, OMNIRAD 819 (0.1 wt. % with respect to the weight of the sol), butylated hydroxytoluene (BHT) (0.1 wt. % with respect to the weight of the sol), and isatin (0.05 wt % with respect to the weight of the sol) were dissolved in the sol. The printing sol was then passed through a 1-μm filter.

### Method for Layer-by-Layer 3D Printing

To print objects from ceramic sol with layer-by-layer 3D printing, the following procedure was used. A build tray was assembled with a fluoropolymer release film. Approximately 50 mL of a printing sol was loaded into the build tray at room temperature. Caution was taken to prevent light exposure by performing procedures in a UV-filtered room (yellow lights), or in low-light conditions when UV-filtering was not available. The build platform was abraded with sandpaper and cleaned with IPA as needed. A .STL file was loaded into the software and support structures were applied as required. The settings for printing in the ASIGA MAX-X43 UV stereolithography printer (from Asiga, Sydney, Australia) are listed in Table 4, below.

TABLE 4
Standard settings for build using
the ASIGA MAX-X43 UV printer.

| Setting | Min | Max | Typical | Units |
|---|---|---|---|---|
| Slice Thickness | 0.001 | 0.05 | 0.05 | mm |
| Separation | | | 4 | |
| Distance | 2 | 10 | | mm |
| Separation Velocity | 0.15 | 15 | 1 | mm/s |
| Approach Velocity | .5 | 15 | 2 | mm/s |
| Exposure Time | 0.5 | 45 | 1.5 | s |
| Power | 15 | 30 | 25 | mW/cm² |

After building, the gel sample was immediately removed from the build platform and submerged in two used diethylene glycol monoethyl ether solvent baths followed by one fresh solvent bath. At this point, sol coating was optionally performed as described in Method for Sol Coating. The part was then post-cured in a Clearstone Technologies CA3200 inerted UV cure chamber for 1 minute of 385 nm LED exposure at 20% power under active nitrogen purge. It was then placed in a sealed container until the next step.

### Method for Additive Manufacturing

An alternative approach for additive manufacturing which can be used to fabricate either the gel body from sol A or the surface layer from sol B has been described by Shah et al. (US 2022/0380260 A1). The layer of sol B can be applied to a gel of sol A by placing sol B in the vat of the printer. The gel body comprised of sol A is then raised or lowered into the vat or resin while the appropriate light pattern is projected onto the vat to cure the desired layer of sol B onto gel A. The gel body comprising sol A with a surface of sol B is then removed from the printer.

### Method for Volumetric Additive Manufacturing

If desired, methods for volumetric additive manufacturing as described by Toombs et al (Science 376, Apr. 15, 2022) and Madrid Woolfe et. al (Adv. Sci. 2022, 9, 2105144) and Regehly et al (Nature 2020, Vol 588 p 620-24) and Huffman, et al, Multiphoton Imaging Methods in a Scattering and/or Absorbing Medium, and Articles (US20220350127A1) can also be used. Adjustments to initiator level and light exposure are set to appropriate levels to render components of sol A. A fabricated component of sol A placed in the printer which is then filled with sol B. Light projection patterns to volumetrically render sol B onto the surface of the sol A component are projected. The gel body comprising sol A with a surface of sol B is then removed from the printer.

### Method for Sol Coating

After printing and washing, but before post-curing steps as described in Method for Layer-by-Layer 3D Printing, samples were optionally coated with either a printing sol or modified precursor sol. When a precursor sol was used, OMNIRAD 819 was added at 0.1 wt. %. To coat, the samples were placed on a double layered paper towel with the to-be-coated surface facing up. Sol was loaded into a 3 mL plastic pipette and dripped on to the center of the sample repeatedly until the entire surface was visibly wetted. Excess sol was allowed to flow off the surface. The samples were then moved to another paper towel by carefully lifting with a tongue depressor from the bottom of the sample. The samples were left for about 1 min to allow any excess sol to be absorbed from the edges of the sample. Then post-curing was continued as described in Method for Layer-by-Layer 3D Printing. If both sides of a sample were to be coated, the second side was coated after the post-cure as described here, followed by an additional post-cure step.

### Method for Supercritical Fluid Extraction

The printed gel body was dried via supercritical fluid extraction, e.g., as described in the Method for Supercritical Extraction of Gels in the examples section of WO 2016/191534 A1 (Mayr et al).

### Method for Burnout and Pre-Sinter

The dried gel body was placed on a bed of zirconia beads in an alumina crucible. The crucible was covered with alumina plates and then fired in air according to the following schedule:
1—Heat from 20° C. to 220° C. at 18° C./hour rate,
2—Heat from 220° C. to 244° C. at 1° C./hour rate,
3—Heat from 244° C. to 400° C. at 6° C./hour rate,
4—Heat from 400° C. to 1,020° C. at 60° C./hour rate,
5—Cool from 1,020° C. to 20° C. at 120° C./hour rate.

### Method for Ion Exchange

The pre-sintered body was placed in a 118-ml glass jar containing 1.0N NH₄OH at a depth of about 2.5 cm and soaked for at least 16 hours. The NH₄OH was then poured off and the jar was filled with distilled water. The body was soaked in distilled water for 1 h. The water was then replaced with fresh distilled water. This step was repeated until the pH of the soak water was equal to that of fresh distilled water. The body was then dried at 90-125° C. for a minimum of 15 min.

### Method for Sintering

The pre-sintered, ion-exchanged body was placed on a bed of zirconia beads in an alumina crucible. The crucible was covered with alumina plates, and the sample was sintered in air according to the following schedule:
1—Heat from 20° C. to 1,020° C. at 500° C./hour rate,
2—Heat from 1,020° C. to 1,225° C. at 120° C./hour rate,
3—Hold at 1,225° C. for 2 hours,
4—Cool down from 1,225° C. to 20° C. at 500° C./hour rate.

### Examples 1-4

Ceramic test pieces were printed according to the Method for Layer-by-Layer 3D Printing. At least some parts from each print instance had a coating applied according to the Method of Sol Coating before the post-cure step. Uncoated samples from each print instance are included for comparison. All samples were dried according to the Method for Supercritical Fluid Extraction, and subsequently processed according to the Method for Burnout and Pre-sinter, the Method for Ion Exchange, and the Method for Sintering to form fully dense ceramic articles.

### Example 1 (Ex 1)

Example 1 exhibits prototype veneers printed with printing sol PSol1, with some individual pieces coated on the front or labial side with precursor-sol SPr1. The coated veneers show increased smoothness and a glossy reflection of light. The coating is consistently smooth across the surface of the coated veneers as shown for the 2 samples on the right hand side of FIG. 2, whereas the 2 samples on the left hand side of FIG. 2 show samples which were not coated.

### Example 2 (Ex 2)

Example 2 exhibits prototype veneers printed with printing sol PSol1, with all individual pieces coated on both the front and back sides with different precursor sols. All coated veneers have precursor sol SPr2 on the front and either SPr1 or SPr3 on the back side. SPr1 and SPr3 have different levels of shading dopants, resulting in a subtle color difference between samples. The coated veneers show increased smoothness and a glossy reflection of light. The coating is consistently smooth across the surface of the coated veneers.

### Example 3 (Ex 3)

Example 3 exhibits prototype veneers printed with printing sol PSol2, with some individual pieces coated on the front or labial side. Of the parts with a coating, half were coated with precursor sol SPr1 while the other half were coated with the same sol as used for printing the veneers themselves (PSol2). The coated veneers show increased smoothness and a glossy reflection of light. The coating is consistently smooth across the surface of the coated veneers. Comparing samples with different coating sols, those coated with precursor sol appear more uniformly smooth, while those coated with printing sol display some patches of rougher surface. However, they are still smoother than the uncoated samples from the same set.

Three veneers with the same shape, one of each uncoated, coated with precursor sol, and printing sol, were selected for surface texture analysis, results and comparison of which are found in the Surface Texture Analysis section.

### Example 4 (Ex 4)

Example 4 exhibits prototype veneers printed with printing sol PSol2, with some individual pieces coated on both the front and back sides with the same sol as used for printing: PSol2. The coated veneers show increased smoothness and a glossy reflection of light. The coating is consistently smooth across the surface of the coated veneers.

### Comparative Example 1 (CE1)

Comparative Example 1 is a set of veneers printed with PSol2 without coating in the as-printed state. The veneers were processed through sintering and then delivered to a professional dental technician. Traditional staining and glazing were performed on the samples, resulting in a smooth surface and multiple colors across the labial face of the veneers. One of the veneers, with a shape matching the 3 from Example 3, was analyzed for surface texture, as detailed in the Surface Texture Analysis Section.

### Surface Texture Analysis

Four veneer samples were analyzed with laser confocal microscopy to compare the surface texture and roughness based on different surface processing techniques. The method for this analysis is found in the Method for Surface Texture Analysis section, while results are detailed in Table 5.

TABLE 5

| Sample | Average Roughness (μm) |
|---|---|
| As-sintered (Ex 3) | 1.93 |
| Drip-coated with printing sol (Ex 3) | 0.13 |
| Drip-coated with sol precursor (Ex 3) | 0.16 |
| Hand glazed (CE 1) | 0.36 |

### Gloss Measurement Examples and Analysis

Gloss measurement samples were flat squares 14 mm×14 mm, 1.4 mm thick. They were printed like the other veneer samples, Examples 1-4, with printing sol PSol1 and coated on one side with the same PSol1. The coated sides show increased smoothness and a glossy reflection of light. Gloss measurements show significant increase in gloss on the coated versus uncoated side. The full results are found in Table 6.

TABLE 6

| | |
|---|---|
| Sample 1 | Sample 2 |

| | Untreated | Coated | Untreated | Coated |
|---|---|---|---|---|
| Test | (GU) | (GU) | (GU) | (GU) |
| 1 | 24.6 | 133.7 | 23.9 | 97.9 |
| 2 | 18.5 | 166.0 | 23.9 | 94.8 |
| 3 | 20.6 | 161.7 | 14.8 | 91.8 |
| 4 | 18.1 | 158.7 | 24.6 | 110.0 |
| 5 | 26.5 | 120.1 | 17.7 | 98.6 |
| Mean | 21.7 | 148.0 | 21.0 | 98.6 |
| St Dev | 3.7 | 20.1 | 4.4 | 6.9 |

1. A process of producing a dental zirconia article, the process comprising the steps of
(a) processing a radiation-curable zirconia sol A as construction material in an additive-manufacturing process comprising a radiation-curing step to obtain a zirconia gel article, the zirconia gel article having the shape of a dental article with an outer and an inner surface,
(b) applying a radiation-curable zirconia sol Bₓ on at least a portion of the outer surface of the zirconia gel article,
(c) radiation-curing the radiation-curable zirconia sol Bₓ of step (b),
(d) transforming the zirconia gel article into a zirconia aerogel article,
(e) heat-treating the zirconia aerogel article,
the radiation-curable zirconia sol A being same or different from the radiation-curable zirconia sol Bₓ with respect to chemical composition and/or physical properties.

2. The process according to claim 1, the radiation-curable zirconia sol A and the radiation-curable zirconia sol Bₓ each comprising
crystalline zirconia particles,
a crystal phase stabilizer component,
a radiation-curable component,
a photo-initiator,
a liquid, and
optionally a coloring component,
optionally inhibitor component,
wherein the crystal phase stabilizer component is contained in the crystalline zirconia particles and the optional coloring component can be contained in the crystalline zirconia particles or be present as a separate component.

3. The process according to claim 1, the radiation-curable zirconia sol A and/or the radiation-curable zirconia sol Bₓ being characterized by the following features alone or in combination:
viscosity: less than 500 mPa*s at 23° C.;
showing a transmission of at least 5% at a wavelength of 420 nm for a pathlength of 10 mm;
pH value: 1 to 6.

4. The process according to claim 1, the radiation-curable zirconia sol A differing from the radiation-curable zirconia sol Bₓ with respect to the following features alone or in combination:
color or content of coloring components;
content of crystal phase stabilizer component;
content of crystalline zirconia particles;
viscosity.

5. The process according to claim 1, the processing of the radiation-curable zirconia sol A and the radiation-curing of radiation-curable zirconia sol Bₓ being done at different radiation wavelengths.

6. The process according to claim 1, the transforming step (d) comprising a super-critical extraction step.

7. The process according to claim 1, the applying of the radiation-curable sol Bₓ being done on portions of the outer and inner surface of the zirconia gel article.

8. The process according to claim 1, wherein in step (b) at least two different radiation-curable sols Bₓ are applied.

9. The process according to claim 1, wherein the radiation-curable zirconia sol Bₓ is applied by either of the following methods:
manual coating process;
top-down projection additive-manufacturing process;
volumetric additive-manufacturing process.

10. The process according to claim 1, wherein in step (b)
a radiation-curable sol B₁ is applied to the outer surface of the zirconia gel article, and
a radiation-curable sol B₂ is applied to the inner surface of the zirconia article,
wherein the radiation-curable sol B₂ differs from the radiation-curable sol B₁ by the following properties alone or in combination:
color or content of coloring components,
content of stabilizer component,
content of crystalline zirconia particles,
viscosity.

11. The process according to claim 1 comprising in addition the step of heat-treating the zirconia aerogel article to obtain a calcined dental zirconia article, a pre-sintered dental zirconia article, or a sintered dental zirconia article.

12. A process of producing a dental ceramic zirconia article, the process comprising the following steps:
(a) providing a radiation-curable zirconia sol A comprising nano-sized crystalline zirconia particles, crystal phase stabilizer component, liquid, a radiation-curable component, photo-initiator, optionally coloring component(s) and optionally inhibitor component(s), as described in any of the preceding claims,
(b) processing the radiation-curable zirconia sol A as construction material in an additive manufacturing process to obtain a 3-dim zirconia gel article with an outer and an inner surface,
(c) optionally cleaning the surface of the 3-dim zirconia gel,
(d) applying radiation-curable zirconia sol Bₓ on at least a portion of the outer surface of the 3-dim zirconia gel article,
(e) post-curing the 3-dim zirconia gel article at a temperature in the range of 35 to 80° C. or by additional light curing,
(f) optionally soaking the 3-dim zirconia gel article with another liquid,
(g) transforming the 3-dim zirconia gel article to a 3-dim zirconia aerogel article,
(h) heating the 3-dim zirconia aerogel article to a temperature in the range of 400 to 800° C.,
(i) heating the 3-dim article of the previous step to a temperature in the range of 800 to 1,100° C.,
(j) optionally conducting an ion-exchange step,
(k) applying a sintering step to obtain a 3-dim sintered zirconia ceramic article,
(l) optionally removing support structures remaining from the processing step (b) from the article, if support structures are present, wherein step (l) can be conducted after any of steps (e), (g), (i) or (k).

13. (canceled)

14. The process according to claim 12, wherein the dental ceramic zirconia article with an outer and an inner surface being characterized by the following features in combination:
thickness between outer and inner surface at at least one section: 0.5 to 0.3 mm;
surface roughness of the surface area to which zirconia sol Bₓ has been applied: <0.4 μm;
gloss: at least 50 GU;
opalescence: at least 10.

15. The process according to claim 12, wherein the dental ceramic zirconia article according to any of claims 13 to 14 having the shape of a dental crown, bridge, inlay, onlay, veneer, bracket, buccal tube, cleat or button.

16. The process according to claim 12, wherein in step (b)
a radiation-curable sol B₁ is applied to the outer surface of the zirconia gel article, and
a radiation-curable sol B₂ is applied to the inner surface of the zirconia article,
wherein the radiation-curable sol B₂ differs from the radiation-curable sol B₁ by the following properties alone or in combination:
color or content of coloring components,
content of stabilizer component,
content of crystalline zirconia particles,
viscosity.

17. The process according to claim 12, the radiation-curable zirconia sol A and/or the radiation-curable zirconia sol Bₓ being characterized by the following features alone or in combination:
viscosity: less than 500 mPa*s at 23° C.;
showing a transmission of at least 5% at a wavelength of 420 nm for a pathlength of 10 mm;
pH value: 1 to 6.

18. The process according to claim 12, the radiation-curable zirconia sol A differing from the radiation-curable zirconia sol Bₓ with respect to the following features alone or in combination:
color or content of coloring components;
content of crystal phase stabilizer component;
content of crystalline zirconia particles;
viscosity.

19. The process according to claim 12, the transforming step (g) comprising a super-critical extraction step.

20. The process according to claim 12, the applying of the radiation-curable sol Bₓ being done on portions of the outer and inner surface of the zirconia gel article.

21. The process according to claim 12, wherein the radiation-curable zirconia sol Bₓ is applied by either of the following methods:
manual coating process;
top-down projection additive-manufacturing process;
volumetric additive-manufacturing process.
